(19) United States
(12) Patent Application Publication
(10) Pub. No.: US 2017/0121746 A1
(43) Pub. Date: May 4, 2017

(54) Method For Preparing Nicotinamide Riboside

(71) Applicant: The Procter & Gamble Company, Cincinnati, OH (US)

(72) Inventors: Juan Esteban Velasquez, Cincinnati, OH (US); Phillip Richard Green, Wyoming, OH (US); John August Wos, Mason, OH (US)

(21) Appl. No.: 15/296,083

(22) Filed: Oct. 18, 2016

Related U.S. Application Data
(60) Provisional application No. 62/247,220, filed on Oct. 28, 2015.

(51) Int. Cl. C12P 19/28

(52) U.S. Cl. CPC C12P 19/28

(57) ABSTRACT

A method of making nicotinamide riboside, nicotinamide riboside derivatives, or mixtures thereof is disclosed. The method involves contacting at least the following materials to form a solution: i) α-D-ribose-1-phosphate, α-D-ribose-1-phosphate derivatives, or mixtures thereof; ii) nicotinamide, nicotinamide derivatives, or mixtures thereof; iii) one or more pentosyl transferases (E.C. 2.4.2); iv) and one or more solvents. The resulting solution comprises nicotinamide riboside, nicotinamide riboside derivatives, or mixtures thereof and one or more inorganic orthophosphate anions. The inorganic orthophosphate anions are removed from the solution, leaving a solution of nicotinamide riboside, nicotinamide riboside derivatives, or mixtures thereof.

### FIELD OF THE INVENTION

The present disclosure is directed generally to methods for the synthesis of nicotinamide riboside, nicotinamide riboside derivatives, or mixtures thereof. More specifically, the present disclosure is directed to enzymes and methods to adjust reaction conditions and components in order to maximize the conversion of starting materials towards desired products.

### BACKGROUND OF THE INVENTION

Nicotinamide riboside (NR) has been reported to be active in cosmetic compositions used to treat skin, specifically for general lightening of the skin and of age spots, protection from inflammation, wrinkles, elasticity, and environmental stresses (See US20050267023; US20120022013; WO2015066382). Currently, NR is synthesized utilizing organic chemical methods (WO2007061798, WO201514722). These syntheses are complex, e.g., requiring the addition and removal of protection groups, which adds cost and limits the use of NR in commercial cosmetic products. Thus, there is a need to develop lower cost methods to synthesize NR.

Purine and pyrimidine nucleoside phosphorylases are a class of enzymes that catalyze the reversible phosphorolysis of purine and pyrimidine nucleosides:

Purine/Pyrimidine (Deoxy)Ribonucleoside+Orthophosphate⇄Purine/Pyrimidine+α-D-(Deoxy)Ribose-1-Phosphate

These enzymes, or organisms expressing them, have been utilized to make a variety of nucleosides, and broad substrate recognition has enabled the synthesis of many nucleoside analogs useful as antiviral and anticancer agents (Current Organic Chemistry (2006) 10: 1197-1215). One example is described in WO 2010/055369 A1 which describes the synthesis of 5-methyluridine from guanosine using a purine nucleoside phosphorylase and a pyrimidine nucleoside phosphorylase in a two-step reaction with yields of 80-90% and with productivities of 4.5-14 g/L/h. While NR is not a purine or pyrimidine nucleoside, it is recognized as a substrate by this class of enzymes, which are utilized in vivo to salvage nicotinamide for NAD⁺ production. Thus, most studies have been done demonstrating the phosphorolysis of NR by these enzymes (European Journal of Biochemistry (1997) 243: 408-414; Journal of Biological Chemistry (1951) 193: 497-507). In this case, NR contains a positively charged quaternary nitrogen at neutral pH and nicotinamide is a weak base, so the phosphorolysis reaction releases an H⁺ as shown:

NR⁺+Orthophosphate⇄Nicotinamide+α-D-Ribose-1-Phosphate+H⁺

Although this is theoretically a reversible reaction, at neutral pH and conditions in the cell, the low concentration of H⁺ and presence of orthophosphate thermodynamically drives the reaction to nicotinamide. Lowering the pH helps to drive production of NR, but is not sufficient on its own to achieve the high yields of NR where only a 9% yield is expected at pH 3. Many of the enzymes, such as from beef liver, are not stable at pH 5 or below (Journal of Biological Chemistry (1951) 193: 497-507). Thus, enzymatic synthesis of NR has never been demonstrated with high productivity and yield and there is a need to develop novel pathways, robust enzymes and process conditions to do so.

### SUMMARY OF THE INVENTION

The present invention utilizes available and lower cost starting materials, isolated enzymes, either freely soluble or immobilized for stability and recoverable for reuse, or microorganisms containing said enzymes, and allows for optimization of conditions for each catalytic step to drive the reaction in the preferred direction.

In the first embodiment, the invention provides a pathway (FIG. 1) that is used to convert a ribonucleotide to a ribonucleoside, the ribonucleoside is converted to α-D-ribose-1-phosphate, and α-D-ribose-1-phosphate is converted to nicotinamide riboside.

In a second embodiment, the ribonucleotide of the first embodiment is converted to a ribonucleoside utilizing a phosphoric monoester hydrolase (E.C. 3.1.3) to remove the 5′-monophosphate from the ribonucleotide. Enzymes include, but are not limited to, alkaline phosphatases (E.C. 3.1.3.1), acid phosphatases (E.C. 3.1.3.2) and 5′-nucleotidases (E.C. 3.1.3.5). Examples of these types of enzymes are listed in Table 1. The ribonucleoside is converted to α-D-ribose-1-phosphate, and α-D-ribose-1-phosphate is converted to nicotinamide riboside.

In a third embodiment, the ribonucleoside of embodiments 1 and 2 is contacted with a nucleoside phosphorylase (E.C. 2.4.2.1 or E.C. 2.4.2.2) to produce a free nitrogenous base and α-D-ribose-1-phosphate. Examples of these types of enzymes are listed in Table 1. α-D-Ribose-1-phosphate is converted to nicotinamide riboside.

In a fourth embodiment, the free nitrogenous base is removed from the solution to increase conversion to α-D-Ribose-1-phosphate of the reaction of the third embodiment.

In a fifth embodiment, the free nitrogenous base is removed from the solution by the oxidation of hypoxanthine with xanthine oxidase (E.C. 1.17.3.2) and oxygen, by precipitation due to its low solubility in the solvent (WO 2010/055369 A1), or by adsorbing onto a resin to increase conversion to α-D-Ribose-1-phosphate of the reaction of the third embodiment.

In a sixth embodiment, the α-D-ribose-1-phosphate produced in embodiments 1, 2, 3, 4, and 5 is converted to nicotinamide riboside by the addition of nicotinamide and a pentosyl transferase such as nucleoside phosphorylase (E.C. 2.4.2). Examples of these types of enzymes are listed in Table 1.

In a seventh embodiment, the pH of the reaction of the sixth embodiment is lowered to about 6, or about 5, or about 4, or about 3, or about 2, or about 1 to increase the conversion to nicotinamide riboside.

In an eighth embodiment, the conversion to nicotinamide riboside of the reaction of the sixth embodiment is increased by removing the inorganic orthophosphate product using a phosphorylase to transfer the inorganic orthophosphate to a disaccharide or polysaccharide acceptor. Examples of phosphorylases include, but are not limited to, glycogen phosphorylases (E.C. 2.4.1.1), sucrose phosphorylases (E.C. 2.4.1.7), maltose phosphorylases (E.C. 2.4.1.8), cellobiose phosphorylases (E.C. 2.4.1.20), 1,3-β-oligoglucan phosphorylases (E.C. 2.4.1.30), laminaribiose phosphorylases (E.C. 2.4.1.31), cellodextrin phosphorylases (E.C. 2.4.1.49), α,α-trehalose phosphorylases (E.C. 2.4.1.64 and E.C. 2.4.1.231), 1,3-β-D-glucan phosphorylases (E.C. 2.4.1.97), 1,3-β-galactosyl-N-acetylhexosamine phosphorylases (E.C. 2.4.1.216), trehalose 6-phosphate phosphorylases (E.C. 2.4.1.216), kojibiose phosphorylases (E.C. 2.4.1.230), β-D-galactosyl-(1→4)-L-rhamnose phosphorylases (E.C. 2.4.1.247), nigerose phosphorylases (E.C. 2.4.1.279), N,N′-diacetylchitobiose phosphorylases (E.C. 2.4.1.280), 4-O-β-D-mannosyl-D-glucose phosphorylases (E.C. 2.4.1.281), 3-O-α-D-glucosyl-L-rhamnose phosphorylase (E.C. 2.4.1.282). Examples of these enzymes are listed in Table 1.

In a ninth embodiment, the conversion to nicotinamide riboside of the reaction of the sixth embodiment is increased by precipitating the inorganic orthophosphate product from the solution by the addition of a cation such as Li⁺, Mg²⁺, Ca²⁺, Sr²⁺, Ba²⁺, Mn²⁺, Fe³⁺, Co²⁺, Ni²⁺, Cu²⁺, Ag⁺, Zn²⁺, Cd²⁺, Al³⁺, Pb²⁺, Bi³⁺, and mixtures thereof, such that the phosphate salt obtained has low solubility in water.

In a tenth embodiment, the conversion to nicotinamide riboside of the reaction of the sixth embodiment is increased by binding the inorganic orthophosphate onto an absorbent.

In an eleventh embodiment, the conversion to nicotinamide riboside of the reaction of the sixth embodiment is increased by reacting in solvents, and mixtures of solvents with each other and with water, such that the inorganic orthophosphate has low solubility and is precipitated. Solvent classes include water, alcohols, esters, ethers, ketones, nitriles, amides, sulfoxides, hydrocarbons, chlorinated hydrocarbons, and mixtures thereof. Specific solvents include, but are not limited to, methanol, ethanol, n-propanol, iso-propanol, n-butanol, iso-butanol, sec-butanol, tert-butanol, ethyl acetate, tetrahydrofuran, acetone, methyl ethyl ketone, methyl n-propyl ketone, methyl n-butyl ketone, acetonitrile, dimethylformamide, dimethyl sulfoxide, and mixtures thereof.

In a twelfth embodiment, reaction modifications described in embodiments 7, 8, 9, 10, and 11 may be combined to remove inorganic orthophosphate.

In a thirteenth embodiment, nicotinic acid is reacted under the conditions described in embodiments 6, 7, 8, 9, 10, 11, and 12 to produce nicotinic acid riboside.

In a fourteenth embodiment, the nicotinic acid riboside of embodiment 13 is further amidated to produce nicotinamide riboside. This reaction can be done by using an amidase (E.C. 3.5.1) and an ammonium source such as ammonia, amino acids, and mixtures thereof. Examples of these enzymes are listed in Table 1.

In a fifteenth embodiment, the invention provides a pathway that is used to convert ribose-5-phosphate to α-D-ribose-1-phosphate, and α-D-ribose-1-phosphate is then converted to nicotinamide riboside.

In a sixteenth embodiment, the method of the fifteenth embodiment exposes ribose-5-phosphate to a phosphomutase (E.C. 5.4.2). Examples of phosphopentomutases (E.C. 5.4.2.7), are listed in Table 1.

In a seventeenth embodiment, α-D-ribose-1-phosphate produced in embodiment 16 is converted to nicotinamide riboside by the addition of nicotinamide and a pentosyl transferase such as a nucleoside phosphorylase (E.C. 2.4.2). Examples of these enzymes are listed in Table 1.

In an eighteenth embodiment, the pH of the reaction of the seventeenth embodiment is lowered to about 6, or about 5, or about 4, or about 3, or about 2, or about 1 to increase conversion to nicotinamide riboside.

In a nineteenth embodiment, the conversion to nicotinamide riboside of the reaction of the seventeenth embodiment is increased by removing the inorganic orthophosphate product using a phosphorylase to transfer the inorganic orthophosphate to a disaccharide or polysaccharide acceptor. Examples of phosphorylases include, but are not limited to, glycogen phosphorylases (E.C. 2.4.1.1), sucrose phosphorylases (E.C. 2.4.1.7), maltose phosphorylases (E.C. 2.4.1.8), cellobiose phosphorylases (E.C. 2.4.1.20), 1,3-β-oligoglucan phosphorylases (E.C. 2.4.1.30), laminaribiose phosphorylases (E.C. 2.4.1.31), cellodextrin phosphorylases (E.C. 2.4.1.49), α,α-trehalose phosphorylases (E.C. 2.4.1.64 and E.C. 2.4.1.231), 1,3-β-D-glucan phosphorylases (E.C. 2.4.1.97), 1,3-β-galactosyl-N-acetylhexosamine phosphorylases (E.C. 2.4.1.216), trehalose 6-phosphate phosphorylases (E.C. 2.4.1.216), kojibiose phosphorylases (E.C. 2.4.1.230), β-D-galactosyl-(1→4)-L-rhamnose phosphorylases (E.C. 2.4.1.247), nigerose phosphorylases (E.C. 2.4.1.279), N,N′-diacetylchitobiose phosphorylases (E.C. 2.4.1.280), 4-O-β-D-mannosyl-D-glucose phosphorylases (E.C. 2.4.1.281), 3-O-α-D-glucosyl-L-rhamnose phosphorylase (E.C. 2.4.1.282). Examples of these enzymes are listed in Table 1.

In a twentieth embodiment, the conversion to nicotinamide riboside of the reaction of the seventeenth embodiment is increased by precipitating the inorganic orthophosphate product from solution by the addition of a cation such as Li⁺, Mg²⁺, Ca²⁺, Sr²⁺, Ba²⁺, Mn²⁺, Fe³⁺, Co²⁺, Ni²⁺, Cu²⁺, Ag⁺, Zn²⁺, Cd²⁺, Al³⁺, Pb²⁺, Bi³⁺, and mixtures thereof, such that the phosphate salt obtained has low solubility in water.

In a twenty-first embodiment, the conversion to nicotinamide riboside of the reaction of the seventeenth embodiment is increased by binding the inorganic orthophosphate onto an adsorbent.

In a twenty-second embodiment, the conversion to nicotinamide riboside of the reaction of the seventeenth embodiment is increased by reacting in solvents, and mixtures of solvents with each other and with water, and such that the inorganic orthophosphate has low solubility and is precipitated. Solvent classes include water, alcohols, esters, ethers, ketones, nitriles, amides, sulfoxides, hydrocarbons, chlorinated hydrocarbons, and mixtures thereof. Specific solvents include, but are not limited to, methanol, ethanol, n-propanol, iso-propanol, n-butanol, iso-butanol, sec-butanol, tert-butanol, ethyl acetate, tetrahydrofuran, acetone, methyl ethyl ketone, methyl n-propyl ketone, methyl n-butyl ketone, acetonitrile, dimethylformamide, dimethyl sulfoxide, and mixtures thereof.

In a twenty-third embodiment, reaction modifications described in embodiments 18, 19, 20, 21, and 22 may be combined to remove inorganic orthophosphate.

In a twenty-fourth embodiment, nicotinic acid is reacted under the conditions described in embodiments 17, 18, 19, 20, 21, 22, and 23 to produce nicotinic acid riboside.

In a twenty-fifth embodiment, the nicotinic acid riboside of embodiment 24 is subsequently amidated to produce nicotinamide riboside. This reaction can be done by using an amidase (E.C. 3.5.1) and an ammonium source such as ammonia, amino acid and mixtures thereof. Examples of these enzymes are listed in Table 1.

In a twenty-sixth embodiment, the invention provides a pathway (FIG. 2) that is used to convert a ribonucleotide to 5-phospho-α-D-ribose-1-diphosphate, the 5-phospho-α-D-ribose-1-diphosphate is converted to β-nicotinamide D-ribonucleotide, and the β-nicotinamide D-ribonucleotide is converted to nicotinamide riboside.

In a twenty-seventh embodiment, the ribonucleotide of the twenty-sixth embodiment is contacted with a pentosyl transferase (E.C. 2.4.2) and inorganic diphosphate to produce the free nitrogenous base and 5-phospho-α-D-ribose-1-diphosphate. Examples of these types of enzymes include, but are not limited to, adenine phosphoribosyl transferases (E.C. 2.4.2.7), hypoxanthine phosphoribosyl transferases (E.C. 2.4.2.8), uracil phosphoribosyl transferases (2.4.2.9), orotate phosphoribosyl transferase (E.C. 2.4.2.10), amidophosphoribosyl transferase (E.C. 2.4.2.14), anthranilate phosphoribosyl transferase (2.4.2.18), dioxotetrahydropyrimidine phosphoribosyl transferase (2.4.2.20), xanthine phosphoribosyl transferase (2.4.2.22), and mixtures thereof. Examples of these enzymes are listed in Table 1.

In a twenty-eighth embodiment, the free nitrogenous base from the reaction of the twenty-seventh embodiment is removed from the solution to increase conversion to 5-phospho-α-D-ribose-1-diphosphate.

In a twenty-ninth embodiment, the free nitrogenous from the reaction of the twenty-seventh embodiment is removed from the solution by the oxidation of hypoxanthine with xanthine oxidase (E.C. 1.17.3.2) and oxygen, by precipitation due to its low solubility in water (WO 2010/055369 A1), or by adsorbing onto a resin to increase conversion to 5-phospho-α-D-ribose-1-diphosphate.

In a thirtieth embodiment, the 5-phospho-α-D-ribose-1-diphosphate from embodiments 26, 27, 28, and 29 is contacted with nicotinamide and a pentosyl transferase (E.C. 2.4.2) selected from the group consisting of adenine phosphoribosyl transferases (E.C. 2.4.2.7), hypoxanthine phosphoribosyl transferases (E.C. 2.4.2.8), uracil phosphoribosyl transferases (2.4.2.9), orotate phosphoribosyl transferase (E.C. 2.4.2.10), amidophosphoribosyl transferase (E.C. 2.4.2.14), anthranilate phosphoribosyl transferase (2.4.2.18), dioxotetrahydropyrimidine phosphoribosyl transferase (2.4.2.20), xanthine phosphoribosyl transferase (2.4.2.22), and mixtures thereof to produce β-nicotinamide D-ribonucleotide. Examples of these enzymes are listed in Table 1.

In a thirty-second embodiment, the conversion to β-nicotinamide D-ribonucleotide of the reaction of the thirty-first embodiment is increased by removing the inorganic diphosphate product using an inorganic diphosphatase (E.C. 3.6.1.1). Examples of these enzymes are listed in Table 1.

In a thirty-third embodiment, the conversion to β-nicotinamide D-ribonucleotide of the reaction of the thirty-first embodiment is increased by precipitating the inorganic diphosphate product from solution by the addition of a cation such as Li⁺, Mg²⁺, Ca²⁺, Sr²⁺, Ba²⁺, Mn²⁺, Fe³⁺. Co²⁺, Ni²⁺, Cu²⁺, Ag⁺, Zn²⁺, Cd²⁺, Al³⁺, Pb²⁺, Bi³⁺, and mixtures thereof, such that the phosphate salt obtained has low solubility in water.

In a thirty-fourth embodiment, the conversion to β-nicotinamide D-ribonucleotide of the reaction of the thirty-first embodiment is increased by binding the inorganic diphosphate onto an adsorbent.

In a thirty-fifth embodiment, the conversion to β-nicotinamide D-ribonucleotide of the reaction of the thirty-first embodiment is increased by reacting in solvents, and mixtures of solvents with each other and with water, and such that the inorganic diphosphate has low solubility and is precipitated. Solvent classes include water, alcohols, esters, ethers, ketones, nitriles, amides, sulfoxides, hydrocarbons, chlorinated hydrocarbons, and mixtures thereof. Specific solvents include, but are not limited to, methanol, ethanol, n-propanol, iso-propanol, n-butanol, iso-butanol, sec-butanol, tert-butanol, ethyl acetate, tetrahydrofuran, acetone, methyl ethyl ketone, methyl n-propyl ketone, methyl n-butyl ketone, acetonitrile, dimethylformamide, dimethyl sulfoxide, and mixtures thereof.

In a thirty-sixth embodiment, reaction modifications described embodiments 32, 33, 34, and 35 may be combined to remove diphosphate.

In a thirty-seventh embodiment, nicotinic acid is reacted under the conditions described in embodiments 30, 31, 32, 33, 34, 35, and 36 to produce β-nicotinic acid D-ribonucleotide.

In a thirty-eighth embodiment, the β-nicotinic acid D-ribonucleotide of embodiment 37 is amidated to produce β-nicotinamide D-ribonucleotide. This reaction can be done by using an amidase (E.C. 3.5.1) and an ammonium source such as ammonia, amino acids, and mixtures thereof. Examples of these enzymes are listed in Table 1.

In a thirty-ninth embodiment, the β-nicotinamide D-ribonucleotide of embodiment 30, 31, 32, 33, 34, 35, 36, and 38 is dephosphorylated to produce nicotinamide riboside a phosphoric monoester hydrolase (E.C. 3.1.3). Enzymes include, but are not limited to, alkaline phosphatases (E.C. 3.1.3.1), acid phosphatases (E.C. 3.1.3.2) and 5′-nucleotidases (E.C. 3.1.3.5). Examples of these enzymes are listed in Table 1.

In a fortieth embodiment, the β-nicotinic acid D-ribonucleotide from embodiment 37 is dephosphorylated to produce β-nicotinic acid D-riboside using a phosphoric monoester hydrolase (E.C. 3.1.3). Enzymes include, but are not limited to, alkaline phosphatases (E.C. 3.1.3.1), acid phosphatases (E.C. 3.1.3.2) and 5′-nucleotidases (E.C. 3.1.3.5). Examples of these enzymes are listed in Table 1.

In a forty-first embodiment, the β-nicotinic acid D-riboside from embodiment 40 is amidated to produce nicotinamide riboside. This reaction can be done using an amidase (E.C. 3.5.1) and an ammonium source such as ammonia, amino acid and mixtures thereof. Examples of these enzymes are listed in Table 1.

### BRIEF DESCRIPTION OF THE DRAWINGS

FIG. 1 illustrates the pathway for the conversion of a ribonucleotide to nicotinamide riboside via ribonucleoside and α-D-ribose-1-phosphate intermediates.

FIG. 2 illustrates the pathway for the conversion of a ribonucleotide to nicotinamide riboside via 5-phospho-α-D-ribose-1-diphosphate and β-nicotinamide D-ribonucleotide intermediates.

FIG. 3 illustrates the pathway for the conversion of a ribonucleotide to nicotinamide riboside via D-ribofuranose-5-phosphate and β-nicotinamide D-ribonucleotide intermediates.

FIG. 4 illustrates the pathway for the conversion of a ribonucleotide to nicotinamide riboside via ribonucleoside, α-D-ribose-1-phosphate, and 1,4-dihydronicotinamide riboside intermediates.

FIG. 5 illustrates methods for removing reaction products to increase conversion of reactants to desired intermediates or products.

FIG. 6 illustrates ¹H-NMR spectroscopy data evidencing: a) the production of α-D-ribose-1-phosphate during the nucleoside phosphorylase catalyzed step between inosine and inorganic orthophosphate when hypoxanthine is removed by reacting with oxygen catalyzed by xanthine oxidase and b) the production of nicotinamide riboside during the nucleoside phosphorylase catalyzed step between nicotinamide and α-D-ribose-1-phosphate when inorganic orthophosphate is removed by reacting with sucrose catalyzed by sucrose phosphorylase.

### DETAILED DESCRIPTION OF THE INVENTION

### I. Definitions

As used herein, the term “nucleoside” refers to a glycosylamine having a nitrogenous base, such as a purine or pyrimidine, linked to a 5-carbon sugar (e.g. D-ribose or 2-deoxy-D-ribose) via a β-glycosidic linkage. Nucleosides are also referred as “ribonucleosides” when the sugar moiety is D-ribose and as “deoxyribonucleosides” when the sugar moiety is 2-deoxy-D-ribose.

As used herein, the term “nucleotide”, also known as “nucleoside monophosphate”, refers to a compound having a nucleoside esterified to an orthophosphate group via the hydroxyl group bound to the 5-carbon of the sugar moiety. Nucleotides are also referred as “ribonucleotides” when the sugar moiety is D-ribose and as “deoxyribonucleotides” when the sugar moiety is 2-deoxy-D-ribose.

As used herein, the term “nitrogenous base”, refers to a compound containing a nitrogen atom that has the chemical properties of a base. Non-limiting examples of nitrogenous bases are compounds comprising pyridine, purine, or pyrimidine moieties, including, but not limited to adenine, guanine, hypoxanthine, thymine, cytosine, uracil, and nicotinamide.

As used herein, the term “purine nucleoside” refers to a nucleoside, wherein the nitrogenous base is a purine.

As used herein, the term “pyrimidine nucleoside” refers to a nucleoside, wherein the nitrogenous base is a pyrimidine.

As used herein, the term “pyridine nucleoside” refers to a nucleoside, wherein the nitrogenous base is a pyridine.

As used herein, the term “inorganic orthophosphate” refers to a compound composed of four oxygen atoms arranged in an almost regular tetrahedral array about a central phosphorus atom. Inorganic orthophosphate may be present in several ionic forms, depending on the pH of the solution, including [PO₄]³⁻, [HPO₄]²⁻, [H₂PO₄]⁻, and H₃PO4.

As used herein, the term “inorganic diphosphate”, also known as “inorganic pyrophosphate”, refers to a compound containing one P—O—P bond generated by corner sharing of two PO₄ tetrahedra. Inorganic diphosphate may be present in several ionic forms, depending on the pH of the solution, including [P₂O₇]⁴⁻, [HP₂O₇]³⁻, [H₂P₂O₇]²⁻, [H₃P₂O₇]⁻, and H₄P₂O₇.

As used herein, the term “conservative substitution” refers to the substitution in a polypeptide of an amino acid with a functionally similar amino acid. A conservative substitution involves replacement of an amino acid residue with an amino acid residue having a similar side chain. Families of amino acid residues having similar side chains have been defined within the art, and include amino acids with basic side chains (e.g., lysine, arginine, and histidine), acidic side chains (e.g., aspartic acid and glutamic acid), uncharged polar side chains (e.g., glycine, asparagine, glutamine, serine, threonine, tyrosine, and cysteine), nonpolar side chains (e.g., alanine, valine, leucine, isoleucine, proline, phenylalanine, methionine, and tryptophan), β-branched side chains (e.g., threonine, valine, and isoleucine) and aromatic side chains (e.g., tyrosine, phenylalanine, tryptophan, and histidine).

As used herein, the term “converting” refers to a chemical transformation from one molecule to another, primarily catalyzed by an enzyme or enzymes, although other organic or inorganic catalysts may be used.

As used herein, the term “conversion,” in the context of chemical transformations, refers to the ratio in % between the molar amount of the desired product and the molar amount of the limiting reagent.

As used herein, the term “endogenous” refers to polynucleotides, polypeptides, or other compounds that are expressed naturally or originate within an organism or cell. That is, endogenous polynucleotides, polypeptides, or other compounds are not exogenous. For instance, an “endogenous” polynucleotide or peptide is present in the cell when the cell was originally isolated from nature.

As used herein, the term “exogenous” refers to any polynucleotide or polypeptide that is not naturally found or expressed in the particular cell or organism where expression is desired. Exogenous polynucleotides, polypeptides, or other compounds are not endogenous.

As used herein, the term “identical” or percent “identity,” in the context of two or more polynucleotide or polypeptide sequences, refers to two or more sequences that are the same or have a specified percentage of nucleotides or amino acid residues that are the same, when compared and aligned for maximum correspondence, as measured using sequence comparison algorithms or by visual inspection.

As used herein, the term “isolated enzyme” refers to enzymes free of a living organism. Isolated enzymes of the invention may be suspended in solution following lysing of the cell they were expressed in, partially or highly purified, soluble or bound to an insoluble matrix.

As used herein, the term “naturally-occurring” refers to an object that can be found in nature. For example, a polypeptide or polynucleotide sequence that is present in an organism (including viruses) that can be isolated from a source in nature and which has not been intentionally modified by man in the laboratory is naturally-occurring. As used herein, “naturally-occurring” and “wild-type” are synonyms.

As used herein, a recombinant gene that is “over-expressed” produces more RNA and/or protein than a corresponding naturally-occurring gene in the microorganism. Methods of measuring amounts of RNA and protein are known in the art. Over-expression can also be determined by measuring protein activity such as enzyme activity. Depending on the embodiment of the invention, “over-expression” is an amount at least 3%, at least 5%, at least 10%, at least 20%, at least 25%, or at least 50% more. An over-expressed polynucleotide is generally a polynucleotide native to the host cell, the product of which is generated in a greater amount than that normally found in the host cell. Over-expression is achieved by, for instance and without limitation, operably linking the polynucleotide to a different promoter than the polynucleotide's native promoter or introducing additional copies of the polynucleotide into the host cell.

As used herein, the term “polynucleotide” refers to a polymer composed of nucleotides. The polynucleotide may be in the form of a separate fragment or as a component of a larger nucleotide sequence construct, which has been derived from a nucleotide sequence isolated at least once in a quantity or concentration enabling identification, manipulation, and recovery of the sequence and its component nucleotide sequences by standard molecular biology methods, for example, using a cloning vector. When a nucleotide sequence is represented by a DNA sequence (i.e., A, T, G, C), this also includes an RNA sequence (i.e., A, U, G, C) in which “U” replaces “T.” Put another way, “polynucleotide” refers to a polymer of nucleotides removed from other nucleotides (a separate fragment or entity) or can be a component or element of a larger nucleotide construct, such as an expression vector or a polycistronic sequence. Polynucleotides include DNA, RNA and cDNA sequences.

As used herein, the term “polypeptide” refers to a polymer composed of amino acid residues which may or may not contain modifications such as phosphates and formyl groups.

As used herein, “recombinant polynucleotide” refers to a polynucleotide having sequences that are not joined together in nature. A recombinant polynucleotide may be included in a suitable vector, and the vector can be used to transform a suitable host cell. A host cell that comprises the recombinant polynucleotide is referred to as a “recombinant host cell.” The polynucleotide is then expressed in the recombinant host cell to produce, e.g., a “recombinant polypeptide.”

As used herein, the term “recombinant expression vector” refers to a DNA construct used to express a polynucleotide that, e.g., encodes a desired polypeptide. A recombinant expression vector can include, for example, a transcriptional subunit comprising: i) an assembly of genetic elements having a regulatory role in gene expression, for example, promoters and enhancers; ii) a structural or coding sequence which is transcribed into mRNA and translated into protein; and iii) appropriate transcription and translation initiation and termination sequences. Recombinant expression vectors are constructed in any suitable manner. The nature of the vector is not critical, and any vector may be used, including plasmid, virus, bacteriophage, and transposon. Possible vectors for use in the invention include, but are not limited to, chromosomal, nonchromosomal and synthetic DNA sequences, e.g., bacterial plasmids; phage DNA; yeast plasmids; and vectors derived from combinations of plasmids and phage DNA, DNA from viruses such as vaccinia, adenovirus, fowl pox, baculovirus, SV40, and pseudorabies.

As used herein, a “recombinant gene” is not a naturally-occurring gene. A recombinant gene is man-made. A recombinant gene includes a protein coding sequence operably linked to expression control sequences. Embodiments include, but are not limited to, an exogenous gene introduced into a microorganism, an endogenous protein coding sequence operably linked to a heterologous promoter (i.e., a promoter not naturally linked to the protein coding sequence) and a gene with a modified protein coding sequence (e.g., a protein coding sequence encoding an amino acid change or a protein coding sequence optimized for expression in the microorganism). The recombinant gene is maintained in the genome of the microorganism, on a plasmid in the microorganism or on a phage in the microorganism.

As used herein, the term “substantially homologous” or “substantially identical” in the context of two nucleic acids or polypeptides, generally refers to two or more sequences or subsequences that have at least 40%, 60%, 80%, 90%, 95%, 96%, 97%, 98% or 99% nucleotide or amino acid residue identity, when compared and aligned for maximum correspondence, as measured using sequence comparison algorithms or by visual inspection. The substantial identity can exist over any suitable region of the sequences, such as, for example, a region that is at least about 50 residues in length, a region that is at least about 100 residues, or a region that is at least about 150 residues. In certain embodiments, the sequences are substantially identical over the entire length of either or both comparison biopolymers.

### II. Synthesis of Nicotinamide Riboside with α-D-Ribose-1-Phosphate as Intermediate

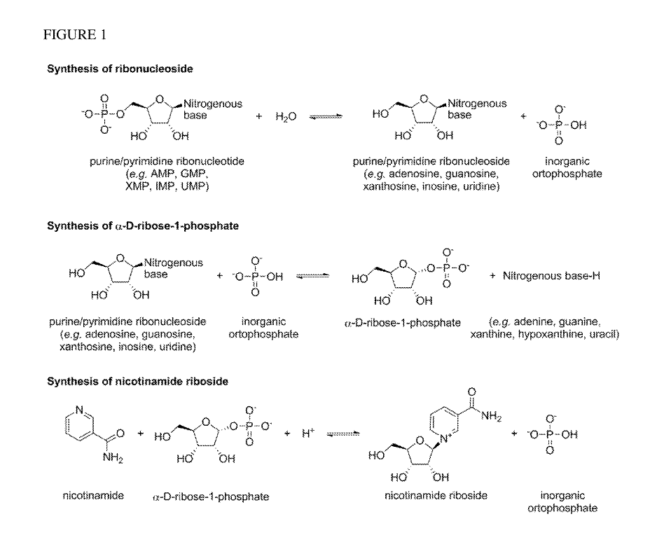

The current invention provides chemical pathways (FIG. 1) and methods to produce pyridine ribosides and its derivatives (e.g. nicotinamide riboside) via α-D-ribose-1-phosphate and its derivatives.

Synthesis of Pyridine Nucleosides, Pyridine Nucleoside Derivatives, or Mixtures Thereof from α-D-Ribose-1-Phosphate, α-D-Ribose-1-Phosphate Derivatives, or Mixtures Thereof

In one embodiment, a method of making pyridine nucleosides, pyridine nucleoside derivatives, or mixtures thereof is provided. The method comprises the steps of:
a) contacting at least at least the following materials to form a solution:
   i. α-D-ribose-1-phosphate, α-D-ribose-1-phosphate derivatives, or mixtures thereof,
   ii. one or more pyridines, pyridine derivatives, or mixtures thereof,
   iii. one or more pentosyl transferases (E.C. 2.4.2), and
   iv. one or more solvents;
   wherein the solution comprises pyridine ribosides, pyridine riboside derivatives, or mixtures thereof and one or more inorganic orthophosphate anions; further, wherein said one or more inorganic orthophosphate anions are selected from the group consisting of [H₂PO₄]⁻ and [HPO₄]²⁻; and
b) removing said one or more inorganic orthophosphate anions from said solution.

In one embodiment, the method of making pyridine nucleosides, pyridine nucleoside derivatives, or mixtures thereof comprises the steps of:
a) contacting at least the following materials to form a solution:
   i. α-D-ribose-1-phosphate, α-D-ribose-1-phosphate derivatives, or mixtures thereof,
   ii. one or more pyridines, pyridine derivatives, or mixtures thereof,
   iii. one or more pentosyl transferases (E.C. 2.4.2), and
   iv. one or more solvents;
   wherein the solution comprises pyridine ribosides, pyridine riboside derivatives, or mixtures thereof and one or more inorganic orthophosphate anions; further, wherein said one or more inorganic orthophosphate anions are selected from the group consisting of [H₂PO₄]⁻ and [HPO₄]²⁻; and
b) removing said pyridine ribosides, pyridine riboside derivatives, or mixtures thereof from said solution.

In one embodiment, the method of making pyridine nucleosides, pyridine nucleoside derivatives, or mixtures thereof comprises the steps of:
a) contacting at least the following materials to form a solution:
   i. α-D-deoxyribose-1-phosphate, α-D-deoxyribose-1-phosphate derivatives, or mixtures thereof,
   ii. one or more pyridines, pyridine derivatives, or mixtures thereof,
   iii. one or more pentosyl transferases (E.C. 2.4.2), and
   iv. one or more solvents;
   wherein the solution comprises pyridine deoxyribosides, pyridine deoxyriboside derivatives, or mixtures thereof and one or more inorganic orthophosphate anions; further, wherein said one or more inorganic orthophosphate anions are selected from the group consisting of [H₂PO₄]⁻ and [HPO₄]²⁻; and
b) removing said one or more inorganic orthophosphate anions from said solution.

In one embodiment, the method of making pyridine nucleosides, pyridine nucleoside derivatives, or mixtures thereof comprises the steps of:
a) contacting at least the following materials to form a solution:
   i. α-D-deoxyribose-1-phosphate, α-D-deoxyribose-1-phosphate derivatives, or mixtures thereof,
   ii. one or more pyridines, pyridine derivatives, or mixtures thereof,
   iii. one or more pentosyl transferases (E.C. 2.4.2), and
   iv. one or more solvents;
   wherein the solution comprises pyridine deoxyribosides, pyridine deoxyriboside derivatives, or mixtures thereof and one or more inorganic orthophosphate anions; further, wherein said one or more inorganic orthophosphate anions are selected from the group consisting of [H₂PO₄]⁻ and [HPO₄]²⁻; and
b) removing said pyridine deoxyribosides, pyridine deoxyriboside derivatives, or mixtures thereof from said solution.
   Synthesis of Nicotinamide Riboside, Nicotinamide Riboside Derivatives, or Mixtures Thereof from α-D-Ribose-1-Phosphate, α-D-Ribose-1-Phosphate Derivatives, or Mixtures Thereof

In another embodiment, a method of making nicotinamide riboside, nicotinamide riboside derivatives, or mixtures thereof is provided. The method comprises the steps of:
a) contacting at least the following materials to form a solution:
   i. α-D-ribose-1-phosphate, α-D-ribose-1-phosphate derivatives, or mixtures thereof,
   ii. nicotinamide, nicotinamide derivatives, or mixtures thereof,
   iii. one or more pentosyl transferases (E.C. 2.4.2), and
   iv. one or more solvents;
   wherein the solution comprises nicotinamide riboside, nicotinamide riboside derivatives, or mixtures thereof and one or more inorganic orthophosphate anions; further, wherein said one or more inorganic orthophosphate anions are selected from the group consisting of [H₂PO₄]⁻ and [HPO₄]²⁻; and
b) removing said one or more inorganic orthophosphate anions from said solution.

In another embodiment, the method of making nicotinamide riboside, nicotinamide riboside derivatives, or mixtures thereof comprises the steps of:
a) contacting at least the following materials to form a solution:
   i. α-D-ribose-1-phosphate, α-D-ribose-1-phosphate derivatives, or mixtures thereof,
   ii. nicotinamide, nicotinamide derivatives, or mixtures thereof,
   iii. one or more pentosyl transferases (E.C. 2.4.2), and
   iv. one or more solvents;
   wherein the solution comprises nicotinamide riboside, nicotinamide riboside derivatives, or mixtures thereof and one or more inorganic orthophosphate anions; further, wherein said one or more inorganic orthophosphate anions are selected from the group consisting of [H₂PO₄]⁻ and [HPO₄]²⁻; and
b) removing said nicotinamide riboside, nicotinamide riboside derivatives, or mixtures thereof from said solution.

In the context of the present invention, α-D-ribose-1-phosphate refers to a compound having a ribose sugar (ribofuranose) moiety covalently bound through the 1 carbon to an orthophosphate moiety via α-O-glycosidic bond. α-D-ribose-1-phosphate can be monobasic or dibasic salts of α-D-ribose-1-phosphate, diprotonated α-D-ribose-1-phosphate, any anionic form of α-D-ribose-1-phosphate, or mixtures thereof. Non-limiting examples of salts of α-D-ribose-1-phosphate are α-D-ribose-1-phosphate salts of metallic cations, α-D-ribose-1-phosphate salts of organo-metallic cations, α-D-ribose-1-phosphate salts of ammonium, α-D-ribose-1-phosphate salts of substituted ammonium, α-D-ribose-1-phosphate salts of oxycations, α-D-ribose-1-phosphate salts of organic cations, and α-D-ribose-1-phosphate salts of other cations known by those skilled in the art. Non limiting examples of substituted ammonium are cyclohexylammonium, N-cyclohexylcyclohexanamine, isopropylammonium, ethylenediammonium, sarcosinium, L-histidinium, glycinium, and 4-aminopyridinium. Non limiting examples of oxycations are pervanadyl and vanadyl ions.

α-D-Ribose-1-phosphate derivatives retain the ribofuranose moiety, but alcohols may be derivitized or replaced with other constituents. Non-limiting examples of α-D-ribose-1-phosphate derivatives are O-alkyloxy derivatives of α-D-ribose-1-phosphate, O-aryloxy derivatives of α-D-ribose-1-phosphate, and O-acyloxy derivatives of α-D-ribose-1-phosphate. Non-limiting examples of O-alkyloxy derivatives of α-D-ribose-1-phosphate are O-methoxy derivatives of α-D-ribose-1-phosphate, O-ethoxy derivatives of α-D-ribose-1-phosphate, and O-propoxy derivatives of α-D-ribose-1-phosphate. Non-limiting examples of O-aryloxy derivatives of α-D-ribose-1-phosphate are O-phenoxy derivatives of α-D-ribose-1-phosphate. Non-limiting examples of O-acyloxy derivatives of α-D-ribose-1-phosphate are O-acetoxy derivatives of α-D-ribose-1-phosphate.

Non-limiting example of pyridines are nicotinamide and other compounds containing at least one pyridine functional group. Non-limiting examples of pyridine derivatives are nicotinamide derivatives. Non-limiting examples of nicotinamide derivatives are any ionic form of nicotinamide, nicotinic acid in any of its protonated or ionic forms or its salts, 1,4-dihydronicotinamide in any of its protonated or ionic forms or its salts, 1,4-dihydronicotinic acid in any of its protonated or ionic forms or its salts, N-alkyl derivatives of nicotinamide, N-hydroxyalkyl derivatives of nicotinamide, N-aryl derivatives of nicotinamide, C-hydroxy derivatives of nicotinamide, C-alkoxy derivatives of nicotinamide, and C-halogenated derivatives of nicotinamide.

Non-limiting example of pyridine nucleosides are nicotinamide riboside and other glycosylamines comprising a pyridine functional group with a β-N-glycosidic bond to a carbohydrate.

In the context of the present invention, nicotinamide riboside, also known as niacinamide riboside, 1-(β-D-ribofuranosyl)nicotinamide, nicotinamide-β-riboside, or nicotinamide ribonucleoside, refers to the pyridine nucleoside that consists of a ribose sugar (ribofuranose) moiety covalently bound through the 1 carbon to nicotinamide via β-N-glycosidic bond to the ring nitrogen. Nicotinamide riboside can be any ionic form of nicotinamide riboside, any salt of nicotinamide riboside, or mixtures thereof. Non-limiting examples of salts of nicotinamide riboside are chloride salts of nicotinamide riboside, phosphate salts of nicotinamide riboside, sulfate salts of nicotinamide riboside, carbonate or bicarbonate salts of nicotinamide riboside, and organic acid salts of nicotinamide riboside (e.g. maleates, citrates, malates, formates, succinates, acetates, and tartrates).

Non-limiting examples of pyridine nucleoside derivatives are nicotinamide riboside derivatives. Non-limiting examples of nicotinamide riboside derivatives are 1,4-dihydronicotinamide riboside in any of its protonated or ionic forms or its salts, nicotinic acid riboside in any of its protonated or ionic forms or its salts, 1,4-dihydronicotinic acid riboside in any of its protonated or ionic forms or its salts, N-alkyl derivatives of nicotinamide riboside, N-hydroxyalkyl derivatives of nicotinamide riboside, N-aryl derivatives of nicotinamide riboside, O-alkoxy derivatives of nicotinamide riboside, C-hydroxylated derivatives of nicotinamide riboside, C-alkoxy derivatives of nicotinamide riboside, C-halogenated derivatives of nicotinamide riboside, O-alkyloxy derivatives of nicotinamide riboside, O-aryloxy derivatives of nicotinamide riboside, and O-acyloxy derivatives of nicotinamide riboside.

In one embodiment of the present invention, the pentosyl transferases (E.C. 2.4.2) catalyze a reaction to convert: a) α-D-ribose-1-phosphate, α-D-ribose-1-phosphate derivatives, or mixtures thereof and b) one or more pyridine, pyridine derivatives, or mixtures thereof to pyridine ribosides, pyridine riboside derivatives, or mixtures thereof. In another embodiment, the pentosyl transferases (E.C. 2.4.2) catalyze a reaction to convert a) α-D-ribose-1-phosphate, α-D-ribose-1-phosphate derivatives, or mixtures thereof and b) nicotinamide, nicotinamide derivatives, or mixtures thereof to nicotinamide riboside, nicotinamide riboside derivatives, or mixtures thereof. In another embodiment, said one or more pentosyl transferases (E.C. 2.4.2) are selected from the group comprising purine nucleoside phosphorylases (E.C. 2.4.2.1), pyrimidine nucleoside phosphorylases (E.C 2.4.2.2), uridine phosphorylases (E.C. 2.4.2.3), thymidine phosphorylases (E.C. 2.4.2.4), nucleoside ribosyltransferases (E.C. 2.4.2.5), nucleoside deoxyribosyltransferases (E.C. 2.4.2.6), guanosine phosphorylase (E.C. 2.4.2.15), urate-ribonucleotide phosphorylases (E.C. 2.4.2.16), deoxyuridine phosphorylase (E.C. 2.4.2.23), S-methyl-5′-thioinosine phosphorylase (E.C. 2.4.2.44), and mixtures thereof. In another embodiment, said one or more pentosyl transferases (E.C. 2.4.2) are selected from the group comprising purine nucleoside phosphorylases (E.C. 2.4.2.1), pyrimidine nucleoside phosphorylases (E.C 2.4.2.2), and mixtures thereof. In another embodiment, said one or more pentosyl transferases (E.C. 2.4.2) are selected from the group consisting of purine nucleoside phosphorylases (E.C. 2.4.2.1), and mixtures thereof. Non-limiting examples of pentosyl transferases (E.C. 2.4.2) are listed in Table 1. Exemplary amino acid sequences of Aeropyrum pernix KI and Cellulomonas sp. purine nucleoside phosphorylases known in the art are respectively set out in SEQ ID NO: 1 and SEQ ID NO: 7.

In one embodiment of the present invention, said one or more solvents are selected from the group comprising water, alcohols, esters, ethers, ketones, nitriles, amides, sulfoxides, hydrocarbons, chlorinated hydrocarbons, and mixtures thereof. In another embodiment of the present invention, said one or more solvents are selected from the group comprising water, methanol, ethanol, n-propanol, iso-propanol, n-butanol, iso-butanol, sec-butanol, tert-butanol, ethyl acetate, tetrahydrofuran, acetone, methyl ethyl ketone, methyl n-propyl ketone, methyl n-butyl ketone, acetonitrile, dimethylformamide, dimethyl sulfoxide, and mixtures thereof. In another embodiment, said one or more solvents comprise water. In yet another embodiment, said solvent is water. In another embodiment, said one or more inorganic orthophosphate anions are essentially insoluble in said one or more solvents.

Under equilibrium conditions, the conversion of nicotinamide and α-D-ribose-1-phosphate into nicotinamide riboside is determined by the equilibrium constant (Kₑ) of the chemical reaction:

NAM+R1P+H⁺⇄NR⁺+Pᵢ

wherein NAM represents nicotinamide, R1P represents α-D-ribose-1-phosphate, NR⁺ represents nicotinamide riboside, and Pᵢ represents inorganic orthophosphate. The value of Kₑ has been estimated to be around 10 (Journal of Biological Chemistry (1951) 193: 497-507). Thus, the approximate conversion of nicotinamide into nicotinamide riboside can be calculated using equation (I):

$$\frac{[ {NR}^{+} ]}{[ NAM ]} = ( {K}_{e} \times {10}^{- p H} ) \times \frac{[ R 1 P ]}{[ {P}_{i} ]} \approx ( 1 \times {10}^{- ( p H - 1 )} ) \times \frac{[ R 1 P ]}{[ {P}_{i} ]} \qquad ( I )$$

and the conversion of α-D-ribose-1-phosphate into nicotinamide riboside using equation (II):

$$\frac{[ {NR}^{+} ]}{[ R 1 P ]} = ( {K}_{e} \times {10}^{- p H} ) \times \frac{[ R 1 P ]}{[ {P}_{i} ]} \approx ( 1 \times {10}^{- ( p H - 1 )} ) \times \frac{[ NAM ]}{[ {P}_{i} ]} \qquad ( II )$$

From expressions (I) and (II), it can be inferred that the lower the concentration of inorganic orthophosphate in the solution or the lower the pH of the reaction, the higher the conversion to nicotinamide riboside. Furthermore, an excess of one of the reagents, i.e. nicotinamide or α-D-ribose-1-phosphate, can increase the conversion. Similar expressions can be derived for nicotinamide riboside derivatives.

To maximize the conversion of α-D-ribose-1-phosphate, α-D-ribose-1-phosphate derivatives, or mixtures thereof and nicotinamide, nicotinamide derivatives, or mixtures thereof into nicotinamide riboside, nicotinamide riboside derivatives, or mixtures thereof, said one or more inorganic orthophosphate anions can be removed from said solution. In the context of the present invention, the step of removing a compound from a solution refers to decreasing the concentration of such compound in the solution compared to the concentration under equilibrium conditions before said removing step. The amount of inorganic orthophosphate to be removed depends on the pH of the reaction, and the concentration of the reagents, as exemplified by expressions (I) and (II). Alternatively, said nicotinamide riboside, nicotinamide riboside derivatives, or mixtures thereof can also be removed from said solution.

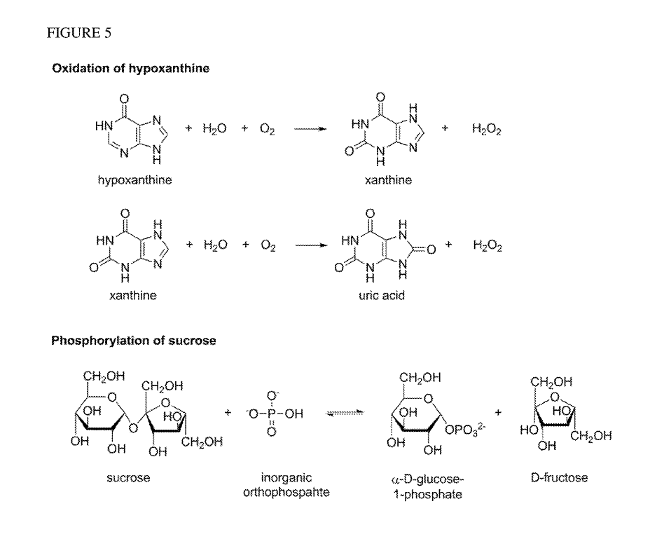

In one embodiment of the present invention, said removing step comprises contacting said one or more inorganic orthophosphate anions with: a) one or more phosphate acceptors, and b) one or more phosphorylases; to produce a phosphate ester (FIG. 5).

In another embodiment, the method of making nicotinamide riboside, nicotinamide riboside derivatives, or mixtures comprises contacting at least the following materials to form a solution:
i. α-D-ribose-1-phosphate, α-D-ribose-1-phosphate derivatives, or mixtures thereof,
ii. nicotinamide, nicotinamide derivatives, or mixtures thereof,
iii. one or more phosphate acceptors,
iv. one or more pentosyl transferases (E.C. 2.4.2),
v. one or more phosphorylases, and
vi. one or more solvents;
   wherein the solution comprises nicotinamide riboside, nicotinamide riboside derivatives, or mixtures thereof.

In one embodiment of the present invention, said one or more phosphate acceptors are selected from the group comprising polysaccharides and disaccharides. In another embodiment, said one or more phosphate acceptors are selected from the group comprising glycogen, sucrose, maltose, cellobiose, 1,3-β-oligoglucan, laminaribiose, cellodextrin, α,α-trehalose, 1,3-β-D-glucan, 1,3-β-galactosyl-N-acetylhexosamine, trehalose 6-phosphate, kojibiose, β-D-galactosyl-(1→4)-L-rhamnose, nigerose, N,N′-diacetylchitobiose, 4-O-β-D-mannosyl-D-glucose phosphorylases, 3-O-α-D-glucosyl-L-rhamnose, and mixtures thereof. In yet another embodiment, said phosphate acceptor is sucrose (FIG. 5).

In another embodiment, said one or more phosphorylases are selected from the group comprising glycogen phosphorylases (E.C. 2.4.1.1), sucrose phosphorylases (E.C. 2.4.1.7), maltose phosphorylases (E.C. 2.4.1.8), cellobiose phosphorylases (E.C. 2.4.1.20), 1,3-β-oligoglucan phosphorylases (E.C. 2.4.1.30), laminaribiose phosphorylases (E.C. 2.4.1.31), cellodextrin phosphorylases (E.C. 2.4.1.49), α,α-trehalose phosphorylases (E.C. 2.4.1.64 and E.C. 2.4.1.231), 1,3-β-D-glucan phosphorylases (E.C. 2.4.1.97), 1,3-β-galactosyl-N-acetylhexosamine phosphorylases (E.C. 2.4.1.216), trehalose 6-phosphate phosphorylases (E.C. 2.4.1.216), kojibiose phosphorylases (E.C. 2.4.1.230), β-D-galactosyl-(1-4)-L-rhamnose phosphorylases (E.C. 2.4.1.247), nigerose phosphorylases (E.C. 2.4.1.279), N,N′-diacetylchitobiose phosphorylases (E.C. 2.4.1.280), 4-O-β-D-mannosyl-D-glucose phosphorylases (E.C. 2.4.1.281), 3-O-α-D-glucosyl-L-rhamnose phosphorylase (E.C. 2.4.1.282), and mixtures thereof. In another embodiment, said one or more phosphorylases are selected from the group consisting of sucrose phosphorylases (E.C. 2.4.1.7), and mixtures thereof. Non-limiting examples of phosphorylases are listed in Table 1. Exemplary amino acid sequences of Streptococcus mutans and Leuconostoc mesenteroides sucrose phosphorylases known in the art are respectively set out in SEQ ID NO: 2 and SEQ ID NO: 8.

In another embodiment, the method of making nicotinamide riboside, nicotinamide riboside derivatives, or mixtures comprises contacting at least the following materials to form a solution:
i. α-D-ribose-1-phosphate, α-D-ribose-1-phosphate derivatives, or mixtures thereof,
ii. nicotinamide, nicotinamide derivatives, or mixtures thereof,
iii. sucrose,
iv. one or more pentosyl transferases (E.C. 2.4.2),
v. one or more sucrose phosphorylases (E.C. 2.4.1.7), and
vi. one or more solvents;
wherein the solution comprises nicotinamide riboside, nicotinamide riboside derivatives, or mixtures thereof.

In one embodiment of the present invention, said removing step comprises contacting said one or more inorganic orthophosphate anions, with one or more cations to produce one or more phosphate salts; wherein said one or more phosphate salts are essentially insoluble in said one or more solvents. In another embodiment of the present invention, said one or more cations are selected from the group comprising Li⁺, Mg²⁺, Ca²⁺, Sr²⁺, Ba²⁺, Mn²⁺, Fe³⁺, Co²⁺, Ni²⁺, Cu²⁺, Ag⁺, Zn²⁺, Cd²⁺, Al³⁺, Pb²⁺, Bi³⁺, and mixtures thereof; and said one or more solvents comprise water. In another embodiment, said one or more cations are selected from the group comprising Mg²⁺, Ca²⁺, Ba²⁺, Fe³⁺, Al³⁺, and mixtures thereof; and said one or more solvents comprise water. In another embodiment, said one or more cations are selected from the group consisting of Mg²⁺, Ca²⁺, Ba²⁺, Fe³⁺, Al³⁺, and mixtures thereof; and said one or more solvents is water.

In another embodiment, the method of making nicotinamide riboside, nicotinamide riboside derivatives, or mixtures comprises contacting at least the following materials to form a solution:
i. α-D-ribose-1-phosphate, α-D-ribose-1-phosphate derivatives, or mixtures thereof,
ii. nicotinamide, nicotinamide derivatives, or mixtures thereof,
iii. one or more pentosyl transferases (E.C. 2.4.2),
iv. one or more cations selected from the group consisting of Mg²⁺, Ca²⁺, Ba²⁺, Fe³⁺, Al³⁺, and mixtures thereof, and
v. water;
   wherein the solution comprises nicotinamide riboside, nicotinamide riboside derivatives, or mixtures thereof.

In another embodiment of the present invention, said removing step comprises adsorbing said one or more inorganic orthophosphate anions onto an adsorbent. Non-limiting examples of adsorbents are ion exchange resins (e.g. anion exchange resins) and chelating resins.

To maximize the conversion of α-D-ribose-1-phosphate, α-D-ribose-1-phosphate derivatives, or mixtures thereof and nicotinamide, nicotinamide derivatives, or mixtures thereof into nicotinamide riboside, nicotinamide riboside derivatives, or mixtures thereof, the reaction can be performed at low pH. In one embodiment of the present invention, said one or more solvents comprise water and the pH of said solution is between about 1 and about 10. In another embodiment, said one or more solvents comprise water and the pH of said solution is between about 2 and about 7. In yet another embodiment, said one or more solvents comprise water and the pH of said solution is between about 3 and about 5.

In another embodiment, the method of making nicotinamide riboside, nicotinamide riboside derivatives, or mixtures comprises contacting at least the following materials to form a solution:
i. α-D-ribose-1-phosphate, α-D-ribose-1-phosphate derivatives, or mixtures thereof,
ii. nicotinamide, nicotinamide derivatives, or mixtures thereof,
iii. sucrose,
iv. one or more pentosyl transferases (E.C. 2.4.2),
v. one or more sucrose phosphorylases (E.C. 2.4.1.7), and
vi. one or more solvents;
   wherein the solution comprises nicotinamide riboside, nicotinamide riboside derivatives, or mixtures thereof; and further, wherein the pH of said solution is between about 2 and about 7.

In another embodiment, the method of making nicotinamide riboside, nicotinamide riboside derivatives, or mixtures comprises contacting at least the following materials to form a solution:
i. α-D-ribose-1-phosphate, α-D-ribose-1-phosphate derivatives, or mixtures thereof,
ii. nicotinamide, nicotinamide derivatives, or mixtures thereof,
iii. one or more pentosyl transferases (E.C. 2.4.2),
iv. one or more cations selected from the group consisting of Fe³⁺, Al³⁺, and mixtures thereof, and
v. water;
   wherein the solution comprises nicotinamide riboside, nicotinamide riboside derivatives, or mixtures thereof; and further, wherein the pH of said solution is between about 2 and about 7.

To control the pH of the reaction, the solution can have a buffer capacity. In another embodiment of the present invention, one or more weak acids or bases are further contacted with said materials to form said solution, wherein said solution has a buffer capacity. Non-limiting examples of weak acids or bases are maleic acid, phosphoric acid, glycine, citric acid, glycylglycine, malic acid, formic acid, succinic acid, acetic acid, pyridine, cacodylic acid, 2-(N-morpholino)ethanesulfonic acid or MES, N-(2-acetamido)iminodiacetic acid or ADA, piperazine-N,N′-bis(2-ethanesulfonic acid) or PIPES, N-(2-acetamido)-2-aminoethanesulfonic acid or ACES, 3-morpholino-2-hydroxypropanesulfonic acid or MOPSO, imidazole, N,N-bis(2-hydroxyethyl)-2-aminoethanesulfonic acid or BES, 3-(N-morpholino)propanesulfonic acid or MOPS, N-(tris(hydroxymethyl)methyl)-2-aminoethanesulfonic acid or TES, N-(2-hydroxyethyl)piperazine-N′-(2-ethanesulfonic acid) or HEPES, 3-(N,N-bis(2-hydroxyethyl)amino)-2-hydroxypropanesulfonic acid or DIPSO, 3-(N-tris(hydroxymethyl)methylamino)-2-hydroxypropanesulfonic acid or TAPSO, triethanolamine or TEA, N-(2-hydroxyethyl)piperazine-N′-(2-hydroxypropanesulfonic acid) or HEPPSO, piperazine-N,N′-bis(2-hydroxypropanesulfonic acid) or POPSO, N-(tri(hydroxymethyl)methyl)glycine or tricine, tris(hydroxymethyl)aminomethane or Tris, N,N-bis(2-hydroxyethyl)glycine (bicine), N-(tris(hydroxymethyl)methyl-3-aminopropanesulfonic acid or TAPS, 2-amino-2-methyl-1,3-propanediol or AMPD, N-(1,1-dimethyl-2-hydroxyethyl)-3-amino-2-hydroxypropanesulfonic acid or AMPSO, 2-aminoethanesulfonic acid or taurine, boric acid, ammonia, N-cyclohexyl-2-aminoethanesulfonic acid or CHES, 2-amino-2-methyl-1-propanol or AMP, N-cyclohexyl-2-hydroxyl-3-aminopropanesulfonic acid or CAPSO, carbonic acid, N-cyclohexyl-3-aminopropanesulfonic acid or CAPS, any or its protonated or ionic forms or salts, and mixtures thereof.

Synthesis of α-D-Ribose-1-Phosphate, α-D-Ribose-1-Phosphate Derivatives, or Mixtures Thereof from Ribonucleosides

The α-D-ribose-1-phosphate, α-D-ribose-1-phosphate derivatives, or mixtures thereof used in some of the methods of making nicotinamide riboside, nicotinamide riboside derivatives, or mixtures thereof provided in the current invention can be synthesized from ribonucleosides. In one embodiment of the present invention, said α-D-ribose-1-phosphate, α-D-ribose-1-phosphate derivatives, or mixtures thereof are produced by the method comprising contacting at least the following materials: a) one or more ribonucleosides, b) one or more inorganic orthophosphate anions, c) one or more pentosyl transferases (E.C. 2.4.2), and d) one or more solvents to form a solution comprising α-D-ribose-1-phosphate, α-D-ribose-1-phosphate derivatives, or mixtures thereof and one or more free nitrogenous bases.

In one embodiment of the present invention, said one or more ribonucleosides are selected from the group comprising purine ribonucleosides. In another embodiment, said one or more ribonucleosides are selected from the group comprising inosine, guanosine, and mixtures thereof. In another embodiment, said one or more ribonucleosides is inosine. In another embodiment, said one or more free nitrogenous bases are one or more purines. In another embodiment, said one or more free nitrogenous bases are selected from the group comprising hypoxanthine, guanine, and mixtures thereof. In yet another embodiment, said one or more free nitrogenous bases is hypoxanthine.

In one embodiment of the present invention, the pentosyl transferases (E.C. 2.4.2) catalyze a reaction to convert: a) one or more ribonucleosides and b) one or more inorganic orthophosphate anions into a) α-D-ribose-1-phosphate, α-D-ribose-1-phosphate derivatives, or mixtures thereof and b) one or more free nitrogenous bases. In another embodiment, said one or more pentosyl transferases (E.C. 2.4.2) are selected from the group comprising purine nucleoside phosphorylases (E.C. 2.4.2.1), pyrimidine nucleoside phosphorylases (E.C 2.4.2.2), uridine phosphorylases (E.C. 2.4.2.3), thymidine phosphorylases (E.C. 2.4.2.4), nucleoside ribosyltransferases (E.C. 2.4.2.5), nucleoside deoxyribosyltransferases (E.C. 2.4.2.6), guanosine phosphorylase (E.C. 2.4.2.15), urate-ribonucleotide phosphorylases (E.C. 2.4.2.16), deoxyuridine phosphorylase (E.C. 2.4.2.23), S-methyl-5′-thioinosine phosphorylase (E.C. 2.4.2.44), and mixtures thereof. In another embodiment, said one or more pentosyl transferases (E.C. 2.4.2) are selected from the group comprising purine nucleoside phosphorylases (E.C. 2.4.2.1), pyrimidine nucleoside phosphorylases (E.C 2.4.2.2), and mixtures thereof. In another embodiment, said one or more pentosyl transferases (E.C. 2.4.2) are selected from the group consisting of purine nucleoside phosphorylases (E.C. 2.4.2.1), and mixtures thereof. In another embodiment, said one or more pentosyl transferases (E.C. 2.4.2) are selected from the group consisting of purine nucleoside phosphorylases (E.C. 2.4.2.1), and mixtures thereof and said one or more ribonucleosides are selected from the group consisting of inosine, guanosine, and mixtures thereof. Non-limiting examples of pentosyl transferases (E.C. 2.4.2) are listed in Table 1. Exemplary amino acid sequences of Aeropyrum pernix KI and Cellulomonas sp. purine nucleoside phosphorylases known in the art are respectively set out in SEQ ID NO: 1 and SEQ ID NO: 7.

In one embodiment of the present invention, said one or more solvents are selected from the group comprising water, alcohols, esters, ethers, ketones, nitriles, amides, sulfoxides, hydrocarbons, chlorinated hydrocarbons, and mixtures thereof. In another embodiment of the present invention, said one or more solvents are selected from the group comprising water, methanol, ethanol, n-propanol, iso-propanol, n-butanol, iso-butanol, sec-butanol, tert-butanol, ethyl acetate, tetrahydrofuran, acetone, methyl ethyl ketone, methyl n-propyl ketone, methyl n-butyl ketone, acetonitrile, dimethylformamide, dimethyl sulfoxide, and mixtures thereof. In another embodiment, said one or more solvents comprise water. In yet another embodiment, said solvent is water.

To maximize the conversion of: a) said one or more ribonucleosides and b) said one or more inorganic orthophosphate anions, into a) said α-D-ribose-1-phosphate, α-D-ribose-1-phosphate derivatives, or mixtures thereof and b) said one or more free nitrogenous bases, the free nitrogenous bases can be removed from said solution. Alternatively, said α-D-ribose-1-phosphate, α-D-ribose-1-phosphate derivatives, or mixtures thereof can also be removed from said solution. In one embodiment, the method of making said α-D-ribose-1-phosphate, α-D-ribose-1-phosphate derivatives, or mixtures thereof further comprise removing said one or more free nitrogenous bases from said solution. In another embodiment of the present invention, said removing step comprises adsorbing said one or more free nitrogenous bases onto an adsorbent. Non-limiting examples of adsorbents are reverse phase or non-polar resins, normal phase or polar resins, ion exchange resins, chelating resins, reversible covalent bond resins, and mixed-mode resins.

In another embodiment, said one or more free nitrogenous bases is hypoxanthine. In another embodiment, the method of making said α-D-ribose-1-phosphate, α-D-ribose-1-phosphate derivatives, or mixtures thereof further comprise contacting said inosine, said one or more inorganic orthophosphate anions, said one or more pentosyl transferases (E.C. 2.4.2), and said one or more solvents with at least one or more xanthine oxidases (E.C. 1.17.3.2) and oxygen, and wherein said one or more solvents comprise water (FIG. 5).

In one embodiment of the present invention, the xanthine oxidases (E.C. 1.17.3.2) catalyze a reaction to convert: a) hypoxanthine, b) oxygen, and c) water, into: a) xanthine, uric acid, or mixtures thereof and b) hydrogen peroxide. Xanthine oxidases (E.C. 1.17.3.2) can be generated from xanthine dehydrogenases (E.C. 1.17.1.4) by methods known by those skilled in the art, including, but not limited to, reversible sulfhydryl oxidation and irreversible proteolytic modification. Non-limiting examples of xanthine oxidases (E.C. 1.17.3.2) and xanthine dehydrogenases (E.C. 1.17.1.4) are listed in Table 1. Exemplary amino acid sequences of Escherichia coli xanthine dehydrogenase/oxidase known in the art are set out in SEQ ID NO: 4, SEQ ID NO: 5, and SEQ ID NO: 6.

In another embodiment, the contacting of said one or more ribonucleosides, said one or more inorganic orthophosphate anions, said one or more pentosyl transferases (E.C. 2.4.2), and said one or more solvents produces one or more essentially insoluble free nitrogenous bases in said one or more solvents.

Synthesis of Ribonucleosides from Ribonucleotides

The ribonucleosides used in some of the methods of making nicotinamide riboside, nicotinamide riboside derivatives, or mixtures thereof provided in the current invention can be synthesized from ribonucleoside phosphates. In one embodiment of the present invention, said one or more ribonucleosides are produced by the method comprising contacting at least the following materials: a) one or more ribonucleoside phosphates, b) water, and c) one or more phosphoric monoester hydrolases (E.C. 3.1.3). In another embodiment of the present invention, said one or more ribonucleosides are produced by the method comprising contacting at least the following materials: a) one or more ribonucleoside phosphates, b) water, c) one or more phosphoric monoester hydrolases (E.C. 3.1.3), and d) one or more solvents.

In another embodiment, said one or more ribonucleoside phosphates are selected from the group comprising ribonucleotides and mixtures thereof. In another embodiment, said one or more ribonucleoside phosphates are selected from the group comprising purine ribonucleotides and mixtures thereof. In yet another embodiment, said one or more ribonucleoside phosphates are selected from the group consisting of inosine 5′-monophosphate, guanosine 5′-monophosphate, and mixtures thereof. In further another embodiment, said one or more ribonucleoside phosphates is inosine 5′-monophosphate.

In one embodiment of the present invention, phosphoric monoester hydrolases (E.C. 3.1.3) catalyze a reaction to convert: a) one or more ribonucleoside phosphates and b) water into one or more ribonucleosides. In another embodiment, said one or more phosphoric monoester hydrolases (E.C. 3.1.3) are selected from the group comprising alkaline phosphatases (E.C. 3.1.3.1), acid phosphatases (E.C. 3.1.3.2), 5′-nucleotidases (E.C. 3.1.3.5), and mixtures thereof. In yet another embodiment, said one or more phosphoric monoester hydrolases (E.C. 3.1.3) are selected from the group consisting of 5′-nucleotidases (E.C. 3.1.3.5). Non-limiting examples of phosphoric monoester hydrolases (E.C. 3.1.3) are listed in Table 1. An exemplary amino acid sequence of Haemophilus influenzae 5′-nucleotidase known in the art is set out in SEQ ID NO: 3.

In one embodiment of the present invention, said one or more solvents are selected from the group comprising water, alcohols, esters, ethers, ketones, nitriles, amides, sulfoxides, hydrocarbons, chlorinated hydrocarbons, and mixtures thereof. In another embodiment of the present invention, said one or more solvents are selected from the group comprising water, methanol, ethanol, n-propanol, iso-propanol, n-butanol, iso-butanol, sec-butanol, tert-butanol, ethyl acetate, tetrahydrofuran, acetone, methyl ethyl ketone, methyl n-propyl ketone, methyl n-butyl ketone, acetonitrile, dimethylformamide, dimethyl sulfoxide, and mixtures thereof. In another embodiment, said one or more solvents comprise water. In yet another embodiment, said solvent is water.

Synthesis of α-D-Ribose-1-Phosphate, α-D-Ribose-1-Phosphate Derivatives, or Mixtures Thereof from D-Ribose-5-Phosphate, D-Ribose-5-Phosphate Derivatives, or Mixtures Thereof

The α-D-ribose-1-phosphate, α-D-ribose-1-phosphate derivatives, or mixtures thereof used in some of the methods of making nicotinamide riboside, nicotinamide riboside derivatives, or mixtures thereof provided in the current invention can be synthesized from D-ribose-5-phosphate, D-ribose-5-phosphate derivatives, or mixtures thereof. In one embodiment of the present invention, said α-D-ribose-1-phosphate, α-D-ribose-1-phosphate derivatives, or mixtures thereof are produced by the method comprising contacting at least the following materials: a) D-ribose-5-phosphate, D-ribose-5-phosphate derivatives, or mixtures thereof, b) one or more phosphomutases (E.C. 5.4.2), and c) one or more solvents.

D-ribose-5-phosphate can be monobasic or dibasic salts of D-ribose-5-phosphate, diprotonated D-ribose-5-phosphate, any anionic form of D-ribose-5-phosphate, or mixtures thereof. Non-limiting examples of salts of D-ribose-5-phosphate are D-ribose-5-phosphate salts of metallic cations, D-ribose-5-phosphate salts of organo-metallic cations, D-ribose-5-phosphate salts of ammonium, D-ribose-5-phosphate salts of substituted ammonium, D-ribose-5-phosphate salts of oxycations, D-ribose-5-phosphate salts of organic cations, and D-ribose-5-phosphate salts of other cations known by those skilled in the art. Non limiting examples of substituted ammonium are cyclohexylammonium, N-cyclohexylcyclohexanamine, isopropylammonium, ethylenediammonium, sarcosinium, L-histidinium, glycinium, and 4-aminopyridinium. Non limiting examples of oxycations are pervanadyl and vanadyl ions.

D-Ribose-5-phosphate derivatives retain the ribofuranose moiety, but alcohols may be derivitized or replaced with other constituents. Non-limiting examples of D-ribose-5-phosphate derivatives are O-alkyloxy derivatives of D-ribose-5-phosphate, O-aryloxy derivatives of D-ribose-5-phosphate, and O-acyloxy derivatives of D-ribose-5-phosphate. Non-limiting examples of O-alkyloxy derivatives of D-ribose-5-phosphate are O-methoxy derivatives of D-ribose-5-phosphate, O-ethoxy derivatives of D-ribose-5-phosphate, and O-propoxy derivatives of D-ribose-5-phosphate. Non-limiting examples of O-aryloxy derivatives of D-ribose-5-phosphate are O-phenoxy derivatives of D-ribose-5-phosphate. Non-limiting examples of O-acyloxy derivatives of D-ribose-5-phosphate are O-acetoxy derivatives of D-ribose-5-phosphate.

In one embodiment of the present invention, the phosphomutases (E.C. 5.4.2) catalyze a reaction to convert: D-ribose-5-phosphate, D-ribose-5-phosphate derivatives, or mixtures thereof into α-D-ribose-1-phosphate, α-D-ribose-1-phosphate derivatives, or mixtures thereof. In another embodiment, said one or more phosphomutases are selected from the group comprising phosphopentomutases (E.C. 5.4.2.7). Non-limiting examples of phosphopentomutases (E.C. 5.4.2.7) are listed in Table 1.

In one embodiment of the present invention, said one or more solvents are selected from the group comprising water, alcohols, esters, ethers, ketones, nitriles, amides, sulfoxides, hydrocarbons, chlorinated hydrocarbons, and mixtures thereof. In another embodiment of the present invention, said one or more solvents are selected from the group comprising water, methanol, ethanol, n-propanol, iso-propanol, n-butanol, iso-butanol, sec-butanol, tert-butanol, ethyl acetate, tetrahydrofuran, acetone, methyl ethyl ketone, methyl n-propyl ketone, methyl n-butyl ketone, acetonitrile, dimethylformamide, dimethyl sulfoxide, and mixtures thereof. In another embodiment, said one or more solvents comprise water. In yet another embodiment, said solvent is water.

Synthesis of D-Ribose-5-Phosphate, D-Ribose-5-Phosphate Derivatives, or Mixtures Thereof from Ribonucleoside Phosphates

The D-ribose-5-phosphate, D-ribose-5-phosphate derivatives, or mixtures thereof used in some of the methods of making nicotinamide riboside, nicotinamide riboside derivatives, or mixtures thereof provided in the current invention can be synthesized from ribonucleoside phosphates. In one embodiment of the present invention, said D-ribose-5-phosphate, D-ribose-5-phosphate derivatives, or mixtures thereof are produced by the method comprising contacting at least the following materials: a) one or more ribonucleoside phosphates, b) water, c) one or more N-glycosyl compound glycosylases (E.C. 3.2.2), and d) one or more solvents.

In another embodiment, said one or more ribonucleoside phosphates are selected from the group comprising ribonucleotides and mixtures thereof. In another embodiment, said one or more ribonucleoside phosphates are selected from the group comprising purine ribonucleotides and mixtures thereof. In yet another embodiment, said one or more ribonucleoside phosphates are selected from the group consisting of inosine 5′-monophosphate, guanosine 5′-monophosphate, and mixtures thereof. In further another embodiment, said one or more ribonucleoside phosphates is inosine 5′-monophosphate.

In one embodiment of the present invention, N-glycosyl compound glycosylases (E.C. 3.2.2) catalyze a reaction to convert: a) one or more ribonucleoside phosphates and b) water into D-ribose-5-phosphate, D-ribose-5-phosphate derivatives, or mixtures thereof. In another embodiment, said one or more N-glycosyl compound glycosylases (E.C. 3.2.2) are selected from the group comprising AMP nucleosidases (E.C. 3.2.2.4), pyrimidine-5′-nucleotide nucleosidases (E.C. 3.2.2.10), inosinate nucleosidases (E.C. 3.2.2.12), and mixtures thereof.

In one embodiment of the present invention, said one or more solvents are selected from the group comprising water, alcohols, esters, ethers, ketones, nitriles, amides, sulfoxides, hydrocarbons, chlorinated hydrocarbons, and mixtures thereof. In another embodiment of the present invention, said one or more solvents are selected from the group comprising water, methanol, ethanol, n-propanol, iso-propanol, n-butanol, iso-butanol, sec-butanol, tert-butanol, ethyl acetate, tetrahydrofuran, acetone, methyl ethyl ketone, methyl n-propyl ketone, methyl n-butyl ketone, acetonitrile, dimethylformamide, dimethyl sulfoxide, and mixtures thereof. In another embodiment, said one or more solvents comprise water. In yet another embodiment, said solvent is water.

Synthesis of Nicotinamide Riboside, Nicotinamide Riboside Derivatives, or Mixtures Thereof from Ribonucleotides

In another embodiment, the method of making nicotinamide riboside, nicotinamide riboside derivatives, or mixtures comprises the following steps:
a) contacting at least the following materials to form a solution:
   i. one or more ribonucleotides,
   ii. one or more phosphoric monoester hydrolases (E.C. 3.1.3)
   iii. water, and
   iv. one or more solvents;
   wherein the solution comprises one or more ribonucleosides;
b) contacting said one or more ribonucleosides with at least the following materials to form a solution:
   i. one or more inorganic orthophosphate anions,
   ii. one or more pentosyl transferases (E.C. 2.4.2), and
   iii. one or more solvents;
   wherein the solution comprises α-D-ribose-1-phosphate, α-D-ribose-1-phosphate derivatives, or mixtures thereof and one or more free nitrogenous bases; and
c) contacting said α-D-ribose-1-phosphate, α-D-ribose-1-phosphate derivatives, or mixtures thereof with at least the following materials to form a solution:
   i. nicotinamide, nicotinamide derivatives, or mixtures thereof,
   ii. one or more phosphate acceptors,
   iii. one or more pentosyl transferases (E.C. 2.4.2),
   iv. one or more phosphorylases, and
   v. one or more solvents;
   wherein the solution comprises nicotinamide riboside, nicotinamide riboside derivatives, or mixtures thereof.

In another embodiment, the method of making nicotinamide riboside, nicotinamide riboside derivatives, or mixtures comprises the following steps:
a) contacting at least the following materials to form a solution:
   i. inosine 5′-monophosphate,
   ii. one or more 5′-nucleotidases (E.C. 3.1.3.5), and
   iii. water;
   wherein the solution comprises inosine;
b) contacting said inosine with at least the following materials to form a solution:
   i. one or more inorganic orthophosphate anions,
   ii. one or more purine nucleoside phosphorylases (E.C. 2.4.2.1),
   iii. one or more xanthine oxidases (E.C. 1.17.3.2),
   iv. oxygen, and
   v. water;
   wherein the solution comprises α-D-ribose-1-phosphate; and
c) contacting said α-D-ribose-1-phosphate with at least the following materials to form a solution:
   i. nicotinamide,
   ii. sucrose,
   iii. one or more purine nucleoside phosphorylases (E.C. 2.4.2.1),
   iv. one or more sucrose phosphorylases (E.C. 2.3.1.7), and
   v. water;
   wherein the solution comprises nicotinamide riboside; and further, wherein the pH of said solution is between about 2 and about 6.

In another embodiment, the method of making nicotinamide riboside, nicotinamide riboside derivatives, or mixtures comprises the following steps:
a) contacting at least the following materials to form a solution:
   i. one or more ribonucleotides,
   ii. one or more phosphoric monoester hydrolases (E.C. 3.1.3),
   iii. water, and
   iv. one or more solvents;
   wherein the solution comprises one or more ribonucleosides;
b) contacting said one or more ribonucleosides with at least the following materials to form a solution:
   i. one or more inorganic orthophosphate anions,
   ii. one or more pentosyl transferases (E.C. 2.4.2), and
   iii. one or more solvents;
   wherein the solution comprises α-D-ribose-1-phosphate, α-D-ribose-1-phosphate derivatives, or mixtures thereof and one or more free nitrogenous bases; and
c) contacting said α-D-ribose-1-phosphate, α-D-ribose-1-phosphate derivatives, or mixtures thereof with at least the following materials to form a solution:
   i. nicotinamide, nicotinamide derivatives, or mixtures thereof,
   ii. one or more cations selected from the group consisting of Mg²⁺, Ca²⁺, Ba²⁺, Fe³⁺, Al³⁺, and mixtures thereof,
   iii. one or more pentosyl transferases (E.C. 2.4.2), and
   iv. water;
   wherein the solution comprises nicotinamide riboside, nicotinamide riboside derivatives, or mixtures thereof.

In another embodiment, the method of making nicotinamide riboside, nicotinamide riboside derivatives, or mixtures comprises the following steps:
a) contacting at least the following materials to form a solution:
   i. inosine 5′-monophosphate,
   ii. one or more 5′-nucleotidases (E.C. 3.1.3.5), and
   iii. water;
   wherein the solution comprises inosine;
b) contacting said inosine with at least the following materials to form a solution:
   i. one or more inorganic orthophosphate anions,
   ii. one or more purine nucleoside phosphorylases (E.C. 2.4.2.1),
   iii. one or more xanthine oxidases (E.C. 1.17.3.2),
   iv. oxygen, and
   v. water;
   wherein the solution comprises α-D-ribose-1-phosphate; and
c) contacting said α-D-ribose-1-phosphate with at least the following materials to form a solution:
   i. nicotinamide,
   ii. one or more cations selected from the group consisting of Fe³⁺, Al³⁺, and mixtures thereof,
   iii. one or more purine nucleoside phosphorylases (E.C. 2.4.2.1), and
   iv. water;
   wherein the solution comprises nicotinamide riboside; and further, wherein the pH of said solution is between about 2 and about 6.

### III. Synthesis of Nicotinamide Riboside with 5-Phospho-α-D-Ribose-1-Diphosphate as Intermediate

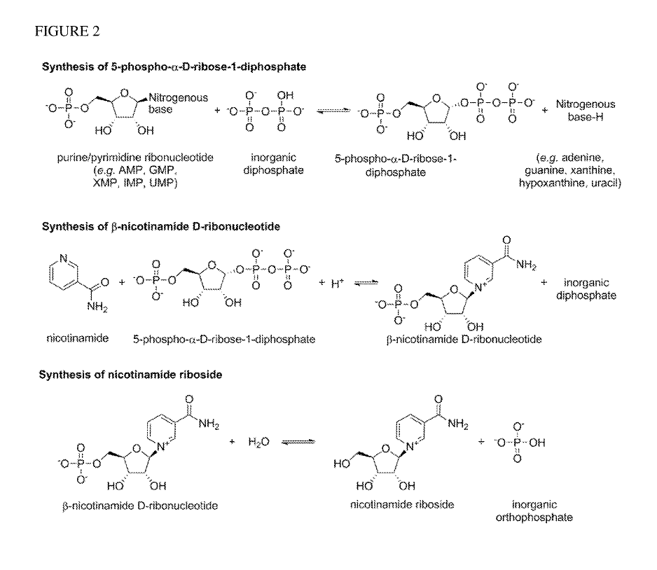

The current invention also provides chemical pathways (FIG. 2) and methods to produce ribonucleosides (e.g. nicotinamide riboside) and its derivatives via 5-phospho-α-D-ribose-1-diphosphate and its derivatives.

Synthesis of Ribonucleosides, Ribonucleoside Derivatives, or Mixtures Thereof from 5-Phospho-α-D-Ribose-1-Diphosphate, 5-Phospho-α-D-Ribose-1-Diphosphate Derivatives, or Mixtures Thereof

In another embodiment, a method of making ribonucleosides, ribonucleoside derivatives, or mixtures thereof is provided. The method comprises the steps of:
a. contacting at least the following materials to form a solution:
   i. 5-phospho-α-D-ribose-1-diphosphate, 5-phospho-α-D-ribose-1-diphosphate derivatives, or mixtures thereof,
   ii. one or more free nitrogenous bases,
   iii. one or more pentosyl transferases (E.C. 2.4.2),
   iv. Mg²⁺, and
   v. one or more solvents;
   wherein the solution comprises one or more ribonucleotides and one or more inorganic diphosphate anions; further, wherein said one or more inorganic diphosphate anions are selected from the group consisting of [HP₂O₇]³⁻, [H₂P₂O₇]²⁻, and [H₃P₂O₇]⁻; and
b. contacting said one or more ribonucleotides with at least the following materials to form a solution:
   i. one or more phosphoric monoester hydrolases (E.C. 3.1.3),
   ii. water, and
   iii. one or more solvents;
   wherein the solution comprises one or more ribonucleosides and one or more inorganic orthophosphate anions; wherein said one or more inorganic orthophosphate anions are selected from the group consisting of [H₂PO₄]⁻ and [HPO₄]²⁻.

In another embodiment, the method of making ribonucleosides, ribonucleoside derivatives, or mixtures comprises contacting at least the following materials to form a solution:
i. 5-phospho-α-D-ribose-1-diphosphate, 5-phospho-α-D-ribose-1-diphosphate derivatives, or mixtures thereof,
ii. one or more nitrogenous bases,
iii. one or more pentosyl transferases (E.C. 2.4.2), and
iv. one or more 5′-nucleotidases (E.C. 3.1.3.5),
v. Mg²⁺,
vi. water, and
vii. one or more solvents;
wherein the solution comprises one or more ribonucleosides, one or more inorganic diphosphate anions, and one or more inorganic orthophosphate anions; further, wherein said one or more inorganic diphosphate anions are selected from the group consisting of [HP₂O₇]³⁻, [H₂P₂O₇]²⁻, and [H₃P₂O₇]⁻; and wherein said one or more inorganic orthophosphate anions are selected from the group consisting of [H₂PO₄]⁻ and [HPO₄]²⁻.

Ribonucleosides are glycosylamines consisting of a nitrogenous base linked to D-ribose via a β-glycosidic linkage. Non-limiting examples of ribonucleosides are purine ribonucleosides, pyrimidine ribonucleosides, and pyridine ribonucleosides. In another embodiment, said one or more ribonucleosides are pyridine ribonucleosides. Non-limiting examples of nitrogenous bases are purine bases, pyrimidine bases, and pyridine bases. Non-limiting examples of purine bases are adenine and its derivatives, guanine and its derivatives, and hypoxanthine and its derivatives. Non-limiting examples of pyrimidine bases are thymine and its derivatives, uracil and its derivatives, and cytosine and its derivatives. Non-limiting examples of pyridine bases are nicotinamide riboside and its derivatives.

The methods described in the current invention can be also used to produce deoxyribonucleosides. In one embodiment, the 5-phospho-α-D-ribose-1-diphosphate, 5-phospho-α-D-ribose-1-diphosphate derivatives, or mixtures thereof are substituted by 5-phospho-α-D-deoxyribose-1-diphosphate, 5-phospho-α-D-deoxyribose-1-diphosphate derivatives, or mixtures thereof.

Synthesis of Nicotinamide Riboside, Nicotinamide Riboside Derivatives, or Mixtures Thereof from 5-Phospho-α-D-Ribose-1-Diphosphate, 5-Phospho-α-D-Ribose-1-Diphosphate Derivatives, or Mixtures Thereof

In another embodiment, a method of making nicotinamide riboside, nicotinamide riboside derivatives, or mixtures thereof is provided. The method comprises the steps of: a. contacting at least the following materials to form a solution:
i. 5-phospho-α-D-ribose-1-diphosphate, 5-phospho-α-D-ribose-1-diphosphate derivatives, or mixtures thereof,
ii. nicotinamide, nicotinamide derivatives, or mixtures thereof,
iii. one or more pentosyl transferases (E.C. 2.4.2),
iv. Mg²⁺, and
v. one or more solvents;
wherein the solution comprises β-nicotinamide D-ribonucleotide, β-nicotinamide D-ribonucleotide derivatives, or mixtures thereof and one or more inorganic diphosphate anions; further, wherein said one or more inorganic diphosphate anions are selected from the group consisting of [HP₂O₇]³⁻, [H₂P₂O₇]²⁻, and [H₃P₂O₇]⁻; and
b. contacting said β-nicotinamide D-ribonucleotide, β-nicotinamide D-ribonucleotide derivatives, or mixtures thereof with at least the following materials to form a solution:
   i. one or more phosphoric monoester hydrolases (E.C. 3.1.3),
   ii. water, and
   iii. one or more solvents;
   wherein the solution comprises nicotinamide riboside, nicotinamide riboside derivatives, or mixtures thereof and one or more inorganic orthophosphate anions; wherein said one or more inorganic orthophosphate anions are selected from the group consisting of [H₂PO₄]⁻ and [HPO₄]²⁻.

In another embodiment, the method of making nicotinamide riboside, nicotinamide riboside derivatives, or mixtures thereof comprises contacting at least the following materials to form a solution:
i. 5-phospho-α-D-ribose-1-diphosphate, 5-phospho-α-D-ribose-1-diphosphate derivatives, or mixtures thereof,
ii. nicotinamide, nicotinamide derivatives, or mixtures thereof,
iii. one or more pentosyl transferases (E.C. 2.4.2), and
iv. one or more 5′-nucleotidases (E.C. 3.1.3.5),
v. Mg²⁺,
vi. water, and
vii. one or more solvents;
wherein the solution comprises nicotinamide riboside, nicotinamide riboside derivatives, or mixtures thereof, one or more inorganic diphosphate anions, and one or more inorganic orthophosphate anions; further, wherein said one or more inorganic diphosphate anions are selected from the group consisting of [HP₂O₇]³⁻, [H₂P₂O₇]²⁻, and [H₃P₂O₇]⁻; and wherein said one or more inorganic orthophosphate anions are selected from the group consisting of [H₂PO₄]⁻ and [HPO₄]²⁻.

5-phospho-α-D-ribose-1-diphosphate can be monobasic, dibasic, tribasic, tetrabasic or pentabasic salts of 5-phospho-α-D-ribose-1-diphosphate, pentaprotonated 5-phospho-α-D-ribose-1-diphosphate, any anionic form of 5-phospho-α-D-ribose-1-diphosphate, or mixtures thereof. Non-limiting examples of salts of 5-phospho-α-D-ribose-1-diphosphate are 5-phospho-α-D-ribose-1-diphosphate salts of metallic cations, 5-phospho-α-D-ribose-1-diphosphate salts of organo-metallic cations, 5-phospho-α-D-ribose-1-diphosphate salts of ammonium, 5-phospho-α-D-ribose-1-diphosphate salts of substituted ammonium, 5-phospho-α-D-ribose-1-diphosphate salts of oxycations, 5-phospho-α-D-ribose-1-diphosphate salts of organic cations, and 5-phospho-α-D-ribose-1-diphosphate salts of other cations known by those skilled in the art. Non limiting examples of substituted ammonium are cyclohexylammonium, N-cyclohexylcyclohexan amine, isopropylammonium, ethylenediammonium, sarcosinium, L-histidinium, glycinium, and 4-aminopyridinium. Non limiting examples of oxycations are pervanadyl and vanadyl ions. Non-limiting examples of 5-phospho-α-D-ribose-1-diphosphate derivatives are O-alkyloxy derivatives of 5-phospho-α-D-ribose-1-diphosphate, O-aryloxy derivatives of 5-phospho-α-D-ribose-1-diphosphate, and O-acyloxy derivatives of 5-phospho-α-D-ribose-1-diphosphate. Non-limiting examples of O-alkyloxy derivatives of 5-phospho-α-D-ribose-1-diphosphate are O-methoxy derivatives of 5-phospho-α-D-ribose-1-diphosphate, O-ethoxy derivatives of 5-phospho-α-D-ribose-1-diphosphate, and O-propoxy derivatives of 5-phospho-α-D-ribose-1-diphosphate. Non-limiting examples of O-aryloxy derivatives of 5-phospho-α-D-ribose-1-diphosphate are O-phenoxy derivatives of 5-phospho-α-D-ribose-1-diphosphate. Non-limiting examples of O-acyloxy derivatives of 5-phospho-α-D-ribose-1-diphosphate are O-acetoxy derivatives of 5-phospho-α-D-ribose-1-diphosphate.

β-Nicotinamide D-ribonucleotide can be any salts of β-nicotinamide D-ribonucleotide, monoprotonated or diprotonated β-nicotinamide D-ribonucleotide and its salts, any ionic form of β-nicotinamide D-ribonucleotide, or mixtures thereof. Non-limiting examples of salts of β-nicotinamide D-ribonucleotide are β-nicotinamide D-ribonucleotide salts of metallic cations, β-nicotinamide D-ribonucleotide of organo-metallic cations, β-nicotinamide D-ribonucleotide salts of ammonium, β-nicotinamide D-ribonucleotide salts of substituted ammonium, β-nicotinamide D-ribonucleotide salts of oxycations, and β-nicotinamide D-ribonucleotide salts of other cations known by those skilled in the art. Non limiting examples of substituted ammonium are cyclohexylammonium, N-cyclohexylcyclohexanamine, isopropylammonium, ethylenediammonium, sarcosinium, L-histidinium, glycinium, and 4-aminopyridinium. Non limiting examples of oxycations are pervanadyl and vanadyl ions. Non-limiting examples of β-nicotinamide D-ribonucleotide derivatives are any ionic form of β-nicotinamide D-ribonucleotide, β-nicotinic acid D-ribonucleotide and its salts, 1,4-dihydronicotinamide D-ribonucleotide, 1,4-dihydronicotinic acid D-ribonucleotide and its salts, N-alkyl derivatives of β-nicotinamide D-ribonucleotide, N-hydroxyalkyl derivatives of β-nicotinamide D-ribonucleotide, N-aryl derivatives of β-nicotinamide D-ribonucleotide, O-alkoxy derivatives of β-nicotinamide D-ribonucleotide, C-hydroxylated derivatives of β-nicotinamide D-ribonucleotide, C-alkoxy derivatives of β-nicotinamide D-ribonucleotide, C-halogenated derivatives of β-nicotinamide D-ribonucleotide, O-alkyloxy derivatives of β-nicotinamide D-ribonucleotide, O-aryloxy derivatives of β-nicotinamide D-ribonucleotide, and O-acyloxy derivatives of β-nicotinamide D-ribonucleotide.

In one embodiment of the present invention, the pentosyl transferases (E.C. 2.4.2) catalyze a reaction to convert: a) 5-phospho-α-D-ribose-1-diphosphate, 5-phospho-α-D-ribose-1-diphosphate derivatives, or mixtures thereof, and b) nicotinamide, nicotinamide derivatives, or mixtures thereof, into a) β-nicotinamide D-ribonucleotide, β-nicotinamide D-ribonucleotide derivatives, or mixtures thereof and b) one or more inorganic diphosphate anions. In another embodiment, said pentosyl transferases (E.C. 2.4.2) are selected from the group comprising nicotinamide phosphoribosyl transferases (E.C. 2.4.2.12). Non-limiting examples of nicotinamide phosphoribosyl transferases (E.C. 2.4.2.12) are listed in Table 1. Exemplary amino acid sequences of Homo sapiens and Synechocystis sp. PCC 6803 nicotinamide phosphoribosyl transferases known in the art are set out respectively in SEQ ID NO: 10 and SEQ ID NO: 12.

In another embodiment of the present invention, the phosphoric monoester hydrolases (E.C. 3.1.3) catalyze a reaction to convert: a) β-nicotinamide D-ribonucleotide, β-nicotinamide D-ribonucleotide derivatives, or mixtures thereof and b) water into: a) nicotinamide riboside, nicotinamide riboside derivatives, or mixtures thereof and b) one or more inorganic orthophosphate anions. In another embodiment, said one or more phosphoric monoester hydrolases (E.C. 3.1.3) are selected from the group comprising alkaline phosphatases (E.C. 3.1.3.1), acid phosphatases (E.C. 3.1.3.2), 5′-nucleotidases (E.C. 3.1.3.5), and mixtures thereof.

In yet another embodiment, said one or more phosphoric monoester hydrolases (E.C. 3.1.3) are selected from the group consisting of 5′-nucleotidases (E.C. 3.1.3.5). Non-limiting examples of phosphoric monoester hydrolases (E.C. 3.1.3) are listed in Table 1. An exemplary amino acid sequence of Haemophilus influenzae 5′-nucleotidase known in the art is set out in SEQ ID NO: 3.

In another embodiment of the present invention, Mg²⁺ is a cofactor required by the pentosyl transferases (E.C. 2.4.2) and the phosphoric monoester hydrolases (E.C. 3.1.3) to catalyze said reactions. In another embodiment, said Mg²⁺ is substituted for one or more divalent metallic cations. Non-limiting examples of divalent metallic cations are Ba²⁺, Be²⁺, Ca²⁺, Co²⁺, Cu²⁺, Zn²⁺, Mg²⁺, Mn²⁺, Ni²⁺, and Sr²⁺.

In one embodiment of the present invention, said one or more solvents are selected from the group comprising water, alcohols, esters, ethers, ketones, nitriles, amides, sulfoxides, hydrocarbons, chlorinated hydrocarbons, and mixtures thereof. In another embodiment of the present invention, said one or more solvents are selected from the group comprising water, methanol, ethanol, n-propanol, iso-propanol, n-butanol, iso-butanol, sec-butanol, tert-butanol, ethyl acetate, tetrahydrofuran, acetone, methyl ethyl ketone, methyl n-propyl ketone, methyl n-butyl ketone, acetonitrile, dimethylformamide, dimethyl sulfoxide, and mixtures thereof. In another embodiment, said one or more solvents comprise water. In another embodiment, said solvent is water. In yet another embodiment, said one or more inorganic diphosphate anions and said one or more inorganic orthophosphate anions are essentially insoluble in said one or more solvents.

To maximize the conversion of 5-phospho-α-D-ribose-1-diphosphate, 5-phospho-α-D-ribose-1-diphosphate derivatives, or mixtures thereof, and nicotinamide, nicotinamide derivatives, or mixtures thereof into β-nicotinamide D-ribonucleotide, β-nicotinamide D-ribonucleotide derivatives, or mixtures thereof and into nicotinamide riboside, nicotinamide riboside derivatives, or mixtures thereof, said one or more inorganic diphosphate anions can be removed from said solution. In one embodiment of the present invention, the method of making nicotinamide, nicotinamide derivatives, or mixtures thereof further comprises contacting said one or more inorganic diphosphate anions with one or more inorganic diphosphatases (E.C. 3.6.1.1) and water to remove said one or more inorganic diphosphate anions from said solution. Non-limiting examples of inorganic diphosphatases (E.C. 3.6.1.1) are listed in Table 1. An exemplary amino acid sequence of Escherichia coli inorganic diphosphatase known in the art is set out in SEQ ID NO: 11.

In another embodiment, the method of making nicotinamide riboside, nicotinamide riboside derivatives, or mixtures thereof comprises contacting at least the following materials to form a solution:
i. 5-phospho-α-D-ribose-1-diphosphate, 5-phospho-α-D-ribose-1-diphosphate derivatives, or mixtures thereof,
ii. nicotinamide, nicotinamide derivatives, or mixtures thereof,
iii. one or more pentosyl transferases (E.C. 2.4.2),
iv. one or more 5′-nucleotidases (E.C. 3.1.3.5),
v. one or more inorganic diphosphatases (E.C. 3.6.1.1),
vi. Mg²⁺,
vii. water, and
viii. one or more solvents;
   wherein the solution comprises nicotinamide riboside, nicotinamide riboside derivatives, or mixtures thereof, and one or more inorganic orthophosphate anions; further, wherein said one or more inorganic orthophosphate anions are selected from the group consisting of [H₂PO₄]⁻ and [HPO₄]²⁻.

In another embodiment, the method of making nicotinamide, nicotinamide derivatives, or mixtures thereof further comprises contacting said one or more inorganic diphosphate anions with one or more cations to produce one or more diphosphate salts; wherein said one or more diphosphate salts are essentially insoluble in said one or more solvents. In another embodiment, said one or more cations are selected from the group consisting of Li⁺, Mg²⁺, Ca²⁺, Sr²⁺, Ba²⁺, Mn²⁺, Fe³⁺, Co²⁺, Ni²⁺, Cu²⁺, Ag⁺, Zn²⁺, Cd²⁺, Al³⁺, Pb²⁺, Bi³⁺, and mixtures thereof; and said one or more solvents is water. In another embodiment, said one or more cations are selected from the group consisting of Mg²⁺, Ca²⁺, Ba²⁺, Fe³⁺, Al³⁺, and mixtures thereof; and said one or more solvents is water. In yet another embodiment, said one or more cations are selected from the group consisting of Fe³⁺, Al³⁺, and mixtures thereof; and said one or more solvents is water.

In another embodiment, the method of making nicotinamide riboside, nicotinamide riboside derivatives, or mixtures thereof comprises contacting at least the following materials to form a solution:
i. 5-phospho-α-D-ribose-1-diphosphate, 5-phospho-α-D-ribose-1-diphosphate derivatives, or mixtures thereof,
ii. nicotinamide, nicotinamide derivatives, or mixtures thereof,
iii. one or more pentosyl transferases (E.C. 2.4.2),
iv. one or more 5′-nucleotidases (E.C. 3.1.3.5),
v. Mg²⁺,
vi. one or more cations selected from the group consisting of Ca²⁺, Ba²⁺, Fe³⁺, Al³⁺, and mixtures thereof,
vii. water, and
viii. one or more solvents;
   wherein the solution comprises nicotinamide riboside, nicotinamide riboside derivatives, or mixtures thereof.

In another embodiment of the present invention, said removing step comprises contacting said one or more inorganic diphosphate anions with an adsorbent material to remove said one or more inorganic diphosphate anions from said solution. Non-limiting examples of adsorbents are ion exchange resins (e.g. anion exchange resins) and chelating resins.

To maximize the conversion of 5-phospho-α-D-ribose-1-diphosphate, 5-phospho-α-D-ribose-1-diphosphate derivatives, or mixtures thereof, and nicotinamide, nicotinamide derivatives, or mixtures thereof into nicotinamide riboside, nicotinamide riboside derivatives, or mixtures thereof, said one or more inorganic orthophosphate anions can be removed from said solution. In one embodiment of the present invention, the method of making nicotinamide riboside, nicotinamide riboside derivatives, or mixtures thereof further comprising removing said one or more inorganic orthophosphate anions from said solution comprising nicotinamide riboside, nicotinamide riboside derivatives, or mixtures thereof. In another embodiment, said one or more inorganic orthophosphate anions are removed by a method comprising contacting said one or more inorganic orthophosphate anions with at least the following materials: a) one or more phosphate acceptors, and b) one or more phosphorylases; to produce a phosphate ester (FIG. 5).

In one embodiment of the present invention, said one or more phosphate acceptors are selected from the group comprising polysaccharides and disaccharides. In another embodiment, said one or more phosphate acceptors are selected from the group comprising glycogen, sucrose, maltose, cellobiose, 1,3-β-oligoglucan, laminaribiose, cellodextrin, α,α-trehalose, 1,3-β-D-glucan, 1,3-β-galactosyl-N-acetylhexosamine, trehalose 6-phosphate, kojibiose, β-D-galactosyl-(1→4)-L-rhamnose, nigerose, N,N′-diacetylchitobiose, 4-O-β-D-mannosyl-D-glucose phosphorylases, 3-O-α-D-glucosyl-L-rhamnose, and mixtures thereof. In yet another embodiment, said phosphate acceptor is sucrose (FIG. 5).

In another embodiment, said one or more phosphorylases are selected from the group comprising glycogen phosphorylases (E.C. 2.4.1.1), sucrose phosphorylases (E.C. 2.4.1.7), maltose phosphorylases (E.C. 2.4.1.8), cellobiose phosphorylases (E.C. 2.4.1.20), 1,3-β-oligoglucan phosphorylases (E.C. 2.4.1.30), laminaribiose phosphorylases (E.C. 2.4.1.31), cellodextrin phosphorylases (E.C. 2.4.1.49), α,α-trehalose phosphorylases (E.C. 2.4.1.64 and E.C. 2.4.1.231), 1,3-β-D-glucan phosphorylases (E.C. 2.4.1.97), 1,3-β-galactosyl-N-acetylhexosamine phosphorylases (E.C. 2.4.1.216), trehalose 6-phosphate phosphorylases (E.C. 2.4.1.216), kojibiose phosphorylases (E.C. 2.4.1.230), β-D-galactosyl-(1→4)-L-rhamnose phosphorylases (E.C. 2.4.1.247), nigerose phosphorylases (E.C. 2.4.1.279), N,N′-diacetylchitobiose phosphorylases (E.C. 2.4.1.280), 4-O-β-D-mannosyl-D-glucose phosphorylases (E.C. 2.4.1.281), 3-O-α-D-glucosyl-L-rhamnose phosphorylase (E.C. 2.4.1.282), and mixtures thereof. In another embodiment, said one or more phosphorylases are selected from the group consisting of sucrose phosphorylases (E.C. 2.4.1.7), and mixtures thereof. Non-limiting examples of phosphorylases are listed in Table 1. Exemplary amino acid sequences of Streptococcus mutans and Leuconostoc mesenteroides sucrose phosphorylases known in the art are respectively set out in SEQ ID NO: 2 and SEQ ID NO: 8.

In another embodiment, the method of making nicotinamide riboside, nicotinamide riboside derivatives, or mixtures thereof comprises contacting at least the following materials to form a solution:
i. 5-phospho-α-D-ribose-1-diphosphate, 5-phospho-α-D-ribose-1-diphosphate derivatives, or mixtures thereof,
ii. nicotinamide, nicotinamide derivatives, or mixtures thereof,
iii. sucrose,
iv. one or more pentosyl transferases (E.C. 2.4.2), and
v. one or more 5′-nucleotidases (E.C. 3.1.3.5),
vi. one or more sucrose phosphorylases (E.C. 2.4.1.7),
vii. Mg²⁺,
viii. water, and
ix. one or more solvents;
   wherein the solution comprises nicotinamide riboside, nicotinamide riboside derivatives, or mixtures thereof, and one or more inorganic diphosphate anions; further, wherein said one or more inorganic diphosphate anions are selected from the group consisting of [HP₂O₇]³⁻, [H₂P₂O₇]²⁻, and [H₃P₂O₇]⁻.

In one embodiment of the present invention, said one or more inorganic orthophosphate anions are removed by a method comprising contacting said one or more inorganic orthophosphate anions with one or more cations to produce one or more phosphate salts; wherein said one or more phosphate salts are essentially insoluble in said one or more solvents.

In another embodiment of the present invention, said one or more cations are selected from the group comprising Li⁺, Mg²⁺, Ca²⁺, Sr²⁺, Ba²⁺, Mn²⁺, Fe³⁺, Co²⁺, Ni²⁺, Cu²⁺, Ag⁺, Zn²⁺, Cd²⁺, Al³⁺, Pb²⁺, Bi³⁺, and mixtures thereof; and said one or more solvents comprise water. In another embodiment, said one or more cations are selected from the group comprising Mg²⁺, Ca²⁺, Ba²⁺, Fe³⁺, Al³⁺, and mixtures thereof; and said one or more solvents comprise water. In another embodiment, said one or more cations are selected from the group consisting of Mg²⁺, Ca²⁺, Ba²⁺, Fe³⁺, Al³⁺, and mixtures thereof; and said one or more solvents is water.

In another embodiment of the present invention, said one or more inorganic orthophosphate anions are removed by a method comprising adsorbing said one or more inorganic orthophosphate anions onto an adsorbent. Non-limiting examples of adsorbents are ion exchange resins (e.g. anion exchange resins) and chelating resins.

To maximize the conversion of 5-phospho-α-D-ribose-1-diphosphate, 5-phospho-α-D-ribose-1-diphosphate derivatives, or mixtures thereof, and nicotinamide, nicotinamide derivatives, or mixtures thereof into nicotinamide riboside, nicotinamide riboside derivatives, or mixtures thereof, the reaction can be performed at low pH. In one embodiment of the present invention, said one or more solvents comprise water and the pH of said solution is between about 1 and about 10. In another embodiment, said one or more solvents comprise water and the pH of said solution is between about 2 and about 7. In yet another embodiment, said one or more solvents comprise water and wherein the pH of said solution is between about 3 and about 5.

In another embodiment, the method of making nicotinamide riboside, nicotinamide riboside derivatives, or mixtures thereof comprises contacting at least the following materials to form a solution:
i. 5-phospho-α-D-ribose-1-diphosphate, 5-phospho-α-D-ribose-1-diphosphate derivatives, or mixtures thereof,
ii. nicotinamide, nicotinamide derivatives, or mixtures thereof,
iii. one or more pentosyl transferases (E.C. 2.4.2),
iv. one or more 5′-nucleotidases (E.C. 3.1.3.5),
v. one or more inorganic diphosphatases (E.C. 3.6.1.1),
vi. Mg²⁺,
vii. water, and
viii. one or more solvents;
   wherein the solution comprises nicotinamide riboside, nicotinamide riboside derivatives, or mixtures thereof, and one or more inorganic orthophosphate anions; wherein said one or more inorganic orthophosphate anions are selected from the group consisting of [H₂PO₄]⁻ and [HPO₄]²⁻; and further, wherein the pH of said solution is between about 2 and about 7.

In another embodiment, the method of making nicotinamide riboside, nicotinamide riboside derivatives, or mixtures thereof comprises contacting at least the following materials to form a solution:
i. 5-phospho-α-D-ribose-1-diphosphate, 5-phospho-α-D-ribose-1-diphosphate derivatives, or mixtures thereof,
ii. nicotinamide, nicotinamide derivatives, or mixtures thereof,
iii. one or more pentosyl transferases (E.C. 2.4.2),
iv. one or more 5′-nucleotidases (E.C. 3.1.3.5),
v. Mg²⁺,
vi. one or more cations selected from the group consisting of Fe³⁺, Al³⁺, and mixtures thereof,
vii. water, and
viii. one or more solvents;
   wherein the solution comprises nicotinamide riboside, nicotinamide riboside derivatives, or mixtures thereof; and further, wherein the pH of said solution is between about 2 and about 7.

To control the pH of the reaction, the solution can have a buffer capacity. In another embodiment of the present invention, one or more weak acids or bases are further contacted with said materials to form said solution, wherein said solution has a buffer capacity. Non-limiting examples of weak acids or bases are maleic acid, phosphoric acid, glycine, citric acid, glycylglycine, malic acid, formic acid, succinic acid, acetic acid, pyridine, cacodylic acid, 2-(N-morpholino)ethanesulfonic acid or MES, N-(2-acetamido)iminodiacetic acid or ADA, piperazine-N,N′-bis(2-ethanesulfonic acid) or PIPES, N-(2-acetamido)-2-aminoethanesulfonic acid or ACES, 3-morpholino-2-hydroxypropanesulfonic acid or MOPSO, imidazole, N,N-bis(2-hydroxyethyl)-2-aminoethanesulfonic acid or BES, 3-(N-morpholino)propanesulfonic acid or MOPS, N-(tris(hydroxymethyl)methyl)-2-aminoethanesulfonic acid or TES, N-(2-hydroxyethyl)piperazine-N′-(2-ethanesulfonic acid) or HEPES, 3-(N,N-bis(2-hydroxyethyl)amino)-2-hydroxypropanesulfonic acid or DIPSO, 3-(N-tris(hydroxymethyl)methylamino)-2-hydroxypropanesulfonic acid or TAPSO, triethanolamine or TEA, N-(2-hydroxyethyl)piperazine-N′-(2-hydroxypropanesulfonic acid) or HEPPSO, piperazine-N,N′-bis(2-hydroxypropanesulfonic acid) or POPSO, N-(tri(hydroxymethyl)methyl)glycine or tricine, tris(hydroxymethyl)aminomethane or Tris, N,N-bis(2-hydroxyethyl)glycine (bicine), N-(tris(hydroxymethyl)methyl-3-aminopropanesulfonic acid or TAPS, 2-amino-2-methyl-1,3-propanediol or AMPD, N-(1,1-dimethyl-2-hydroxyethyl)-3-amino-2-hydroxypropanesulfonic acid or AMPSO, 2-aminoethanesulfonic acid or taurine, boric acid, ammonia, N-cyclohexyl-2-aminoethanesulfonic acid or CHES, 2-amino-2-methyl-1-propanol or AMP, N-cyclohexyl-2-hydroxyl-3-aminopropanesulfonic acid or CAPSO, carbonic acid, N-cyclohexyl-3-aminopropanesulfonic acid or CAPS, any or its protonated or ionic forms or salts, and mixtures thereof.

Synthesis of 5-Phospho-α-D-Ribose-1-Diphosphate, 5-Phospho-α-D-Ribose-1-Diphosphate Derivatives, or Mixtures Thereof from Ribonucleotides

The 5-phospho-α-D-ribose-1-diphosphate, 5-phospho-α-D-ribose-1-diphosphate derivatives, or mixtures thereof used in some of the methods of making nicotinamide riboside, nicotinamide riboside derivatives, or mixtures thereof provided in the current invention can be synthesized from ribonucleotides. In one embodiment of the present invention, said 5-phospho-α-D-ribose-1-diphosphate, 5-phospho-α-D-ribose-1-diphosphate derivatives, or mixtures thereof are produced by a method comprising contacting at least the following materials:
a) one or more ribonucleotides,
b) one or more inorganic diphosphate anions,
c) Mg²⁺,
d) one or more pentosyl transferases (E.C. 2.4.2), and
e) one or more solvents.

In another embodiment, said one or more ribonucleotides are selected from the group comprising purine ribonucleotides and mixtures thereof. In yet another embodiment, said one or more ribonucleotides are selected from the group consisting of inosine 5′-monophosphate, guanosine 5′-monophosphate, and mixtures thereof. In further another embodiment, said one or more ribonucleotides is inosine 5′-monophosphate.

In one embodiment of the present invention, pentosyl transferases (E.C. 2.4.2) catalyze a reaction to convert: a) one or more ribonucleotides and b) one or more inorganic diphosphate anions, into: a) 5-phospho-α-D-ribose-1-diphosphate, 5-phospho-α-D-ribose-1-diphosphate derivatives, or mixtures thereof and b) one or more free nitrogenous bases. In another embodiment, said one or more pentosyl transferases (E.C. 2.4.2) are selected from the group comprising adenine phosphoribosyl transferases (E.C. 2.4.2.7), hypoxanthine phosphoribosyl transferases (E.C. 2.4.2.8), uracil phosphoribosyl transferases (2.4.2.9), orotate phosphoribosyl transferase (E.C. 2.4.2.10), amidophosphoribosyl transferase (E.C. 2.4.2.14), anthranilate phosphoribosyl transferase (2.4.2.18), dioxotetrahydropyrimidine phosphoribosyl transferase (2.4.2.20), xanthine phosphoribosyl transferase (2.4.2.22), and mixtures thereof. In another embodiment, said one or more ribonucleotides is inosine 5′-monophosphate and said one or more pentosyl transferases are hypoxanthine phosphoribosyl transferases (E.C. 2.4.2.8). Non-limiting examples of pentosyl transferases (E.C. 2.4.2) are listed in Table 1. Exemplary amino acid sequences of Escherichia coli hypoxanthine phosphoribosyl transferase known in the art is set out in SEQ ID NO: 9.

In another embodiment of the present invention, Mg²⁺ is a cofactor required by the pentosyl transferases (E.C. 2.4.2) to catalyze said reactions. In another embodiment, said Mg²⁺ is substituted for one or more divalent metallic cations. Non-limiting examples of divalent metallic cations are Ba²⁺, Be²⁺, Ca²⁺, Co²⁺, C²⁺, Zn²⁺, Mg²⁺, Mn²⁺, Ni²⁺, and Sr²⁺, In one embodiment of the present invention, said one or more solvents are selected from the group comprising water, alcohols, esters, ethers, ketones, nitriles, amides, sulfoxides, hydrocarbons, chlorinated hydrocarbons, and mixtures thereof. In another embodiment of the present invention, said one or more solvents are selected from the group comprising water, methanol, ethanol, n-propanol, iso-propanol, n-butanol, iso-butanol, sec-butanol, tert-butanol, ethyl acetate, tetrahydrofuran, acetone, methyl ethyl ketone, methyl n-propyl ketone, methyl n-butyl ketone, acetonitrile, dimethylformamide, dimethyl sulfoxide, and mixtures thereof. In another embodiment, said one or more solvents comprise water. In another embodiment, said solvent is water.

To maximize the conversion of: a) said one or more ribonucleotides and b) said one or more inorganic diphosphate anions, into a) said 5-phospho-α-D-ribose-1-diphosphate, 5-phospho-α-D-ribose-1-diphosphate derivatives, or mixtures thereof and b) said one or more free nitrogenous bases, the free nitrogenous bases can be removed from said solution. Alternatively, the 5-phospho-α-D-ribose-1-diphosphate, 5-phospho-α-D-ribose-1-diphosphate derivatives, or mixtures thereof can also be removed from said solution. In one embodiment, the method of making said 5-phospho-α-D-ribose-1-diphosphate, 5-phospho-α-D-ribose-1-diphosphate derivatives, or mixtures thereof further comprise removing said one or more free nitrogenous bases from said solution. In another embodiment of the present invention, said removing step comprises adsorbing said one or more free nitrogenous bases onto an adsorbent. Non-limiting examples of adsorbents are reverse phase or non-polar resins, normal phase or polar resins, ion exchange resins, chelating resins, reversible covalent bond resins, and mixed-mode resins.

In another embodiment, said one or more free nitrogenous bases is hypoxanthine. In another embodiment, the method of making said 5-phospho-α-D-ribose-1-diphosphate, 5-phospho-α-D-ribose-1-diphosphate derivatives, or mixtures thereof further comprise contacting said inosine 5′-monophosphate, said one or more inorganic diphosphate anions, said one or more pentosyl transferases (E.C. 2.4.2), and said one or more solvents with at least one or more xanthine oxidases (E.C. 1.17.3.2) and oxygen, and wherein said one or more solvents comprise water (FIG. 5).

In one embodiment of the present invention, the xanthine oxidases (E.C. 1.17.3.2) catalyze a reaction to convert: a) hypoxanthine, b) oxygen, and c) water, into: a) xanthine, uric acid, or mixtures thereof and b) hydrogen peroxide. Xanthine oxidases (E.C. 1.17.3.2) can be generated from xanthine dehydrogenases (E.C. 1.17.1.4) by methods known by those skilled in the art, including, but not limited to, reversible sulfhydryl oxidation and irreversible proteolytic modification. Non-limiting examples of xanthine oxidases (E.C. 1.17.3.2) and xanthine dehydrogenases (E.C. 1.17.1.4) are listed in Table 1. Exemplary amino acid sequences of Escherichia coli xanthine dehydrogenase/oxidase known in the art are set out in SEQ ID NO: 4, SEQ ID NO: 5, and SEQ ID NO: 6.

In another embodiment, the contacting of said one or more ribonucleotides, said one or more inorganic diphosphate anions, said one or more pentosyl transferases (E.C. 2.4.2), and said one or more solvents produces one or more essentially insoluble free nitrogenous bases in said one or more solvents.

Synthesis of Nicotinamide Riboside, Nicotinamide Riboside Derivatives, or Mixtures Thereof from Ribonucleotides

In another embodiment, the method of making nicotinamide riboside, nicotinamide riboside derivatives, or mixtures thereof comprises the steps of:
a. contacting at least the following materials to form a solution:
   i. one or more ribonucleotides,
   ii. one or more inorganic diphosphate anions,
   iii. Mg²⁺,
   iv. one or more pentosyl transferases (E.C. 2.4.2), and
   v. one or more solvents;
   wherein the solution comprises 5-phospho-α-D-ribose-1-diphosphate, 5-phospho-α-D-ribose-1-diphosphate derivatives, or mixtures thereof;
b. contacting said 5-phospho-α-D-ribose-1-diphosphate, 5-phospho-α-D-ribose-1-diphosphate derivatives, or mixtures thereof with at least the following materials to form a solution:
   i. nicotinamide, nicotinamide derivatives, or mixtures thereof,
   ii. one or more pentosyl transferases (E.C. 2.4.2),
   iii. Mg²⁺, and
   iv. one or more solvents;
   wherein the solution comprises β-nicotinamide D-ribonucleotide, β-nicotinamide D-ribonucleotide derivatives, or mixtures thereof; and
c. contacting said β-nicotinamide D-ribonucleotide, β-nicotinamide D-ribonucleotide derivatives, or mixtures thereof with at least the following materials to form a solution:
   i. one or more phosphoric monoester hydrolases (E.C. 3.1.3),
   ii. water, and
   iii. one or more solvents;
   wherein the solution comprises nicotinamide riboside, nicotinamide riboside derivatives, or mixtures thereof.

In another embodiment, the method of making nicotinamide riboside, nicotinamide riboside derivatives, or mixtures thereof comprises the steps of:
a. contacting at least the following materials to form a solution:
   i. inosine 5′-monophosphate,
   ii. one or more inorganic diphosphate anions,
   iii. Mg²⁺,
   iv. one or more hypoxanthine phosphoribosyl transferases (E.C. 2.4.2.8),
   v. one or more xanthine oxidases (E.C. 1.17.3.2),
   vi. oxygen, and
   vii. water;
   wherein the solution comprises 5-phospho-α-D-ribose-1-diphosphate;
b. contacting said 5-phospho-α-D-ribose-1-diphosphate, 5-phospho-α-D-ribose-1-diphosphate derivatives, or mixtures thereof with at least the following materials to form a solution:
   i. nicotinamide,
   ii. one or more nicotinamide phosphoribosyl transferases (E.C. 2.4.2.12),
   iii. one or more inorganic diphosphatases (E.C. 3.6.1.1),
   iv. Mg²⁺, and
   v. water;
   wherein the solution comprises β-nicotinamide D-ribonucleotide, β-nicotinamide D-ribonucleotide derivatives, or mixtures thereof; and
c. contacting said β-nicotinamide D-ribonucleotide, β-nicotinamide D-ribonucleotide derivatives, or mixtures thereof with at least the following materials to form a solution:
   i. one or more 5′-nucleotidases (E.C. 3.1.3.5), and
   ii. water;
   wherein the solution comprises nicotinamide riboside.

### IV. Synthesis of Nicotinamide Riboside with D-Ribofuranose-5-Phosphate as Intermediate

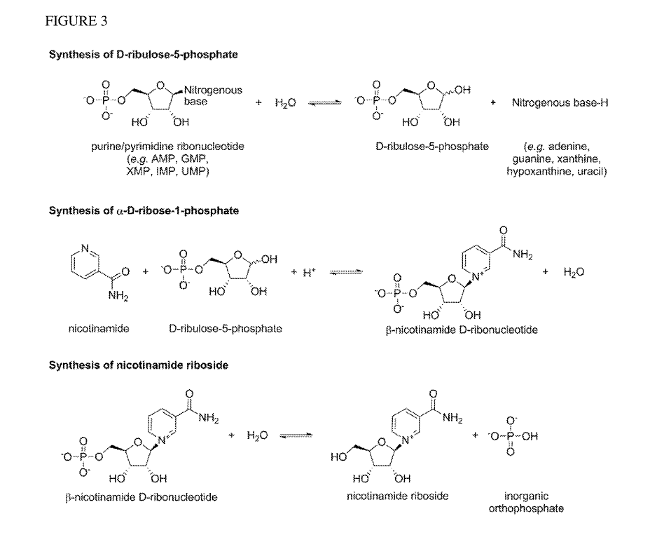

The current invention also provides chemical pathways (FIG. 3) and methods to produce ribonucleosides and its derivatives (e.g. nicotinamide riboside) via D-ribofuranose-5-phosphate and its derivatives.

Synthesis of Nicotinamide Riboside, Nicotinamide Riboside Derivatives, or Mixtures Thereof from D-Ribofuranose-5-Phosphate, D-Ribofuranose-5-Phosphate Derivatives, or Mixtures Thereof

In another embodiment, a method of making nicotinamide riboside, nicotinamide riboside derivatives, or mixtures thereof is provided. The method comprises the steps of: a. contacting at least the following materials to form a solution:
i. D-ribofuranose-5-phosphate, D-ribofuranose-5-phosphate derivatives, or mixtures thereof,
ii. nicotinamide, nicotinamide derivatives, or mixtures thereof,
iii. one or more NMR nucleosidases (E.C. 3.2.2.14), and
iv. one or more solvents;
wherein the solution comprises β-nicotinamide D-ribonucleotide, β-nicotinamide D-ribonucleotide derivatives, or mixtures thereof; and
b. contacting said β-nicotinamide D-ribonucleotide, β-nicotinamide D-ribonucleotide derivatives, or mixtures thereof with at least the following materials to form a solution:
   i. one or more phosphoric monoester hydrolases (E.C. 3.1.3),
   ii. water, and
   iii. one or more solvents;
   wherein the solution comprises nicotinamide riboside, nicotinamide riboside derivatives, or mixtures thereof and one or more inorganic orthophosphate anions; wherein said one or more inorganic orthophosphate anions are selected from the group consisting of [H₂PO₄]⁻ and [HPO₄]²⁻.

In another embodiment, the method of making nicotinamide riboside, nicotinamide riboside derivatives, or mixtures thereof comprises contacting at least the following materials to form a solution:
i. D-ribofuranose-5-phosphate, D-ribofuranose-5-phosphate derivatives, or mixtures thereof,
ii. nicotinamide, nicotinamide derivatives, or mixtures thereof,
iii. one or more NMR nucleosidases (E.C. 3.2.2.14),
iv. one or more 5′-nucleotidases (E.C. 3.1.3.5),
v. water, and
vi. one or more solvents;
wherein the solution comprises nicotinamide riboside, nicotinamide riboside derivatives, or mixtures thereof, and one or more inorganic orthophosphate anions; further, wherein said one or more inorganic orthophosphate anions are selected from the group consisting of [H₂PO₄]⁻ and [HPO₄]²⁻.

D-ribofuranose-5-phosphate can be monobasic or dibasic salts of D-ribofuranose-5-phosphate, diprotonated D-ribofuranose-5-phosphate, any anionic form of D-ribofuranose-5-phosphate, or mixtures thereof. Non-limiting examples of salts of D-ribofuranose-5-phosphate are D-ribofuranose-5-phosphate salts of metallic cations, D-ribofuranose-5-phosphate salts of organo-metallic cations, D-ribofuranose-5-phosphate salts of ammonium, D-ribofuranose-5-phosphate salts of substituted ammonium, D-ribofuranose-5-phosphate salts of oxycations, D-ribofuranose-5-phosphate salts of organic cations, and D-ribofuranose-5-phosphate salts of other cations known by those skilled in the art. Non limiting examples of substituted ammonium are cyclohexylammonium, N-cyclohexylcyclohexanamine, isopropylammonium, ethylenediammonium, sarcosinium, L-histidinium, glycinium, and 4-aminopyridinium. Non limiting examples of oxycations are pervanadyl and vanadyl ions.

D-Ribofuranose-5-phosphate derivatives retain the ribofuranose moiety, but alcohols may be derivitized or replaced with other constituents. Non-limiting examples of D-ribofuranose-5-phosphate derivatives are O-alkyloxy derivatives of D-ribofuranose-5-phosphate, O-aryloxy derivatives of D-ribofuranose-5-phosphate, and O-acyloxy derivatives of D-ribofuranose-5-phosphate. Non-limiting examples of O-alkyloxy derivatives of D-ribofuranose-5-phosphate are O-methoxy derivatives of D-ribofuranose-5-phosphate, O-ethoxy derivatives of D-ribofuranose-5-phosphate, and O-propoxy derivatives of D-ribofuranose-5-phosphate. Non-limiting examples of O-aryloxy derivatives of D-ribofuranose-5-phosphate are O-phenoxy derivatives of D-ribofuranose-5-phosphate. Non-limiting examples of O-acyloxy derivatives of D-ribofuranose-5-phosphate are O-acetoxy derivatives of D-ribofuranose-5-phosphate.

In one embodiment of the present invention, the NMR nucleosidases (E.C. 3.2.2.14) catalyze a reaction to convert: a) D-ribofuranose-5-phosphate, D-ribofuranose-5-phosphate derivatives, or mixtures thereof, and b) nicotinamide, nicotinamide derivatives, or mixtures thereof, into a) β-nicotinamide D-ribonucleotide, β-nicotinamide D-ribonucleotide derivatives, or mixtures thereof and b) water. Non-limiting examples of NMR nucleosidases (E.C. 3.2.2.14) are listed in Table 1.

In another embodiment of the present invention, the phosphoric monoester hydrolases (E.C. 3.1.3) catalyze a reaction to convert: a) β-nicotinamide D-ribonucleotide, β-nicotinamide D-ribonucleotide derivatives, or mixtures thereof and b) water into: a) nicotinamide riboside, nicotinamide riboside derivatives, or mixtures thereof and b) one or more inorganic orthophosphate anions. In another embodiment, said one or more phosphoric monoester hydrolases (E.C. 3.1.3) are selected from the group comprising alkaline phosphatases (E.C. 3.1.3.1), acid phosphatases (E.C. 3.1.3.2), 5′-nucleotidases (E.C. 3.1.3.5), and mixtures thereof.

In yet another embodiment, said one or more phosphoric monoester hydrolases (E.C. 3.1.3) are selected from the group consisting of 5′-nucleotidases (E.C. 3.1.3.5). Non-limiting examples of phosphoric monoester hydrolases (E.C. 3.1.3) are listed in Table 1. An exemplary amino acid sequence of Haemophilus influenzae 5′-nucleotidase known in the art is set out in SEQ ID NO: 3.

In one embodiment of the present invention, said one or more solvents are selected from the group comprising water, alcohols, esters, ethers, ketones, nitriles, amides, sulfoxides, hydrocarbons, chlorinated hydrocarbons, and mixtures thereof. In another embodiment of the present invention, said one or more solvents are selected from the group comprising water, methanol, ethanol, n-propanol, iso-propanol, n-butanol, iso-butanol, sec-butanol, tert-butanol, ethyl acetate, tetrahydrofuran, acetone, methyl ethyl ketone, methyl n-propyl ketone, methyl n-butyl ketone, acetonitrile, dimethylformamide, dimethyl sulfoxide, and mixtures thereof. In another embodiment, said one or more solvents comprise water. In another embodiment, said solvent is water. In yet another embodiment, said one or more inorganic orthophosphate anions are essentially insoluble in said one or more solvents.

Synthesis of D-Ribofuranose-5-Phosphate, D-Ribofuranose-5-Phosphate Derivatives, or Mixtures Thereof from Ribonucleotides

The D-ribofuranose-5-phosphate, D-ribofuranose-5-phosphate derivatives, or mixtures thereof used in some of the methods of making nicotinamide riboside, nicotinamide riboside derivatives, or mixtures thereof provided in the current invention can be synthesized from ribonucleotides. In one embodiment of the present invention, said α D-ribofuranose-5-phosphate, D-ribofuranose-5-phosphate derivatives, or mixtures thereof are produced by the method comprising contacting at least the following materials: a) one or more ribonucleotides b) water, c) one or more nucleotidases (E.C. 3.2.2), and d) one or more solvents to form a solution comprising D-ribofuranose-5-phosphate, D-ribofuranose-5-phosphate derivatives, or mixtures thereof and one or more free nitrogenous bases.

In one embodiment of the present invention, said one or more ribonucleotides are selected from the group comprising purine ribonucleotides and pyrimidine nucleotides. In another embodiment, said one or more nucleotides are selected from the group comprising inosine 5′-monophosphate, guanosine 5′-monophosphate, and mixtures thereof. In another embodiment, said one or more ribonucleotides is inosine 5′-monophosphate. In another embodiment, said one or more free nitrogenous bases are one or more purines or one or more pyrimidines. In another embodiment, said one or more free nitrogenous bases are selected from the group comprising hypoxanthine, guanine, and mixtures thereof. In yet another embodiment, said one or more free nitrogenous bases is hypoxanthine.

In one embodiment of the present invention, the nucleotidases (E.C. 3.2.2) catalyze a reaction to convert: a) one or more ribonucleotides and b) water; into: a) D-ribofuranose-5-phosphate, D-ribofuranose-5-phosphate derivatives, or mixtures thereof and b) one or more free nitrogenous bases. In another embodiment, said one or more nucleotidases (E.C. 3.2.2) are selected from the group comprising AMP nucleosidases (E.C. 3.2.2.4), pyrimidine-5′-nucleotide nucleosidases (E.C. 3.2.2.10), inosinate nucleosidases (E.C. 3.2.2.12), and mixtures thereof.

Non-limiting examples of nucleotidases (E.C. 3.2.2) are listed in Table 1.

In one embodiment of the present invention, said one or more solvents are selected from the group comprising water, alcohols, esters, ethers, ketones, nitriles, amides, sulfoxides, hydrocarbons, chlorinated hydrocarbons, and mixtures thereof. In another embodiment of the present invention, said one or more solvents are selected from the group comprising water, methanol, ethanol, n-propanol, iso-propanol, n-butanol, iso-butanol, sec-butanol, tert-butanol, ethyl acetate, tetrahydrofuran, acetone, methyl ethyl ketone, methyl n-propyl ketone, methyl n-butyl ketone, acetonitrile, dimethylformamide, dimethyl sulfoxide, and mixtures thereof. In another embodiment, said one or more solvents comprise water. In yet another embodiment, said solvent is water.

Synthesis of Nicotinamide Riboside, Nicotinamide Riboside Derivatives, or Mixtures Thereof from Ribonucleotides

In another embodiment, the method of making nicotinamide riboside, nicotinamide riboside derivatives, or mixtures thereof comprises the steps of:
a. contacting at least the following materials to form a solution:
   i. one or more ribonucleotides,
   ii. water,
   iii. one or more nucleotidases (E.C. 3.2.2), and
   iv. one or more solvents;
   wherein the solution comprises D-ribofuranose-5-phosphate, D-ribofuranose-5-phosphate derivatives, or mixtures thereof;
b. contacting said D-ribofuranose-5-phosphate, D-ribofuranose-5-phosphate derivatives, or mixtures thereof with at least the following materials to form a solution:
   i. nicotinamide, nicotinamide derivatives, or mixtures thereof,
   ii. one or more NMR nucleosidases (E.C. 3.2.2.14), and
   iii. one or more solvents;
   wherein the solution comprises β-nicotinamide D-ribonucleotide, β-nicotinamide D-ribonucleotide derivatives, or mixtures thereof; and
c. contacting said β-nicotinamide D-ribonucleotide, β-nicotinamide D-ribonucleotide derivatives, or mixtures thereof with at least the following materials to form a solution:
   i. one or more phosphoric monoester hydrolases (E.C. 3.1.3),
   ii. water, and
   iii. one or more solvents;
   wherein the solution comprises nicotinamide riboside, nicotinamide riboside derivatives, or mixtures thereof and one or more inorganic orthophosphate anions.

In another embodiment, the method of making nicotinamide riboside, nicotinamide riboside derivatives, or mixtures thereof comprises the steps of:
a. contacting at least the following materials to form a solution:
   i. inosine 5′-monophosphate,
   ii. one or more inosinate nucleosidases (E.C. 3.2.2.12), and
   iii. water;
   wherein the solution comprises D-ribofuranose-5-phosphate;
b. contacting said D-ribofuranose-5-phosphate with at least the following materials to form a solution:
   i. nicotinamide,
   ii. one or more NMR nucleosidases (E.C. 3.2.2.14), and
   iii. water;
   wherein the solution comprises β-nicotinamide D-ribonucleotide; and
c. contacting said β-nicotinamide D-ribonucleotide, β-nicotinamide D-ribonucleotide derivatives, or mixtures thereof with at least the following materials to form a solution:
   i. one or more 5′-nucleotidases (E.C. 3.1.3.5), and
   ii. water;
   wherein the solution comprises nicotinamide riboside.

### V. Synthesis of Nicotinamide Riboside Derivatives

### Synthesis of Nicotinic Acid Riboside and its Derivatives

In one embodiment of the present invention, said nicotinamide, nicotinamide derivatives, or mixtures thereof comprises nicotinic acid, nicotinic acid derivatives, or mixtures thereof and said nicotinamide riboside, nicotinamide riboside derivatives, or mixtures thereof comprises nicotinic acid riboside, nicotinic acid riboside derivatives, or mixtures thereof. The nicotinic acid riboside, nicotinic acid riboside derivatives, or mixtures thereof synthesized using some of the methods provided in the current invention can be further converted to nicotinamide riboside, nicotinamide riboside derivatives, or mixtures thereof. In one embodiment of the present invention, said nicotinic acid riboside, nicotinic acid riboside derivatives, or mixtures thereof are further contacted with one or more ammonium sources and one or more amidases (E.C. 3.5.1). In another embodiment, said ammonium sources are selected from the group comprising ammonia, asparagine, glutamate, other amino acids, and mixtures thereof. In another embodiment, said one or more amidases (E.C. 3.5.1) are selected from the group comprising asparaginases (E.C. 3.5.1.1), glutaminases (E.C. 3.5.1.2), omega-amidases (E.C. 3.5.1.3), amidases (E.C. 3.5.1.4), nicotinamidases (E.C. 3.5.1.19), nicotinamide-nucleotide amidase (E.C. 3.5.1.42), and mixtures thereof. Non-limiting examples of nicotinamide-nucleotide amidase (E.C. 3.5.1.42) are listed in Table 1.

### Synthesis of 1,4-Dihydronicotinamide Riboside and its Derivatives

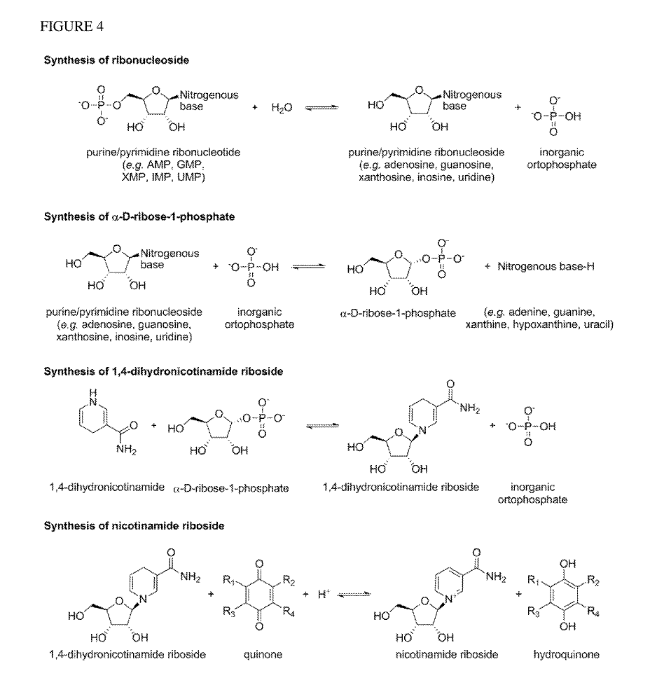

In one embodiment of the present invention, said nicotinamide, nicotinamide derivatives, or mixtures thereof comprises 1,4-dihydronicotinamide, 1,4-dihydronicotinamide derivatives, or mixtures thereof and said nicotinamide riboside, nicotinamide riboside derivatives, or mixtures thereof comprises 1,4-dihydronicotinamide riboside, 1,4-dihydronicotinamide riboside derivatives, or mixtures thereof. The 1,4-dihydronicotinamide riboside, 1,4-dihydronicotinamide riboside derivatives, or mixtures thereof synthesized using some of the methods provided in the current invention can be further converted to nicotinamide riboside, nicotinamide riboside derivatives, or mixtures thereof (FIG. 4). In one embodiment of the present invention, said 1,4-dihydronicotinamide riboside, 1,4-dihydronicotinamide riboside derivatives, or mixtures thereof are further contacted with one or more quinones and one or more ribosyldihydronicotinamide dehydrogenases (E.C. 1.10.99.2, E.C. 1.10.5.1) or one or more NAD(P)H dehydrogenases (E.C. 1.6.5.2) to form a solution comprising nicotinamide riboside, nicotinamide riboside derivatives, or mixtures thereof and one or more hydroquinones.

Alternatively, the nicotinamide riboside, nicotinamide riboside derivatives, or mixtures thereof synthesized using some of the methods provided in the current invention can be further converted to 1,4-dihydronicotinamide riboside, 1,4-dihydronicotinamide riboside derivatives, or mixtures thereof. In one embodiment of the present invention, said nicotinamide riboside, nicotinamide riboside derivatives, or mixtures thereof are further contacted with one or more hydroquinones and one or more ribosyldihydronicotinamide dehydrogenases (E.C. 1.10.99.2, E.C. 1.10.5.1) or one or more NAD(P)H dehydrogenases (E.C. 1.6.5.2) to form a solution comprising 1,4-dihydronicotinamide riboside, 1,4-dihydronicotinamide riboside derivatives, or mixtures thereof and one or more quinones. In another embodiment of the present invention, said nicotinamide riboside, nicotinamide riboside derivatives, or mixtures thereof are further contacted with a reducing reagent to form a solution comprising 1,4-dihydronicotinamide riboside, 1,4-dihydronicotinamide riboside derivatives, or mixtures thereof.

Non-limiting examples of quinones are co-enzyme Q, menadione, 3-hydroxy-1-methylindoline-5,6-dione, 17β-17-hydroxyestr-1(10)-ene-3,4-dione, 2,6-dichloroindophenol, 5-(aziridin-1-yl)-2,4-dinitrobenzamide, benzo(a)pyrene-3,6-quinone, methyl red, estrone-3,4-quinone, and mytocin C. Non-limiting examples of hydroquinones are reduced co-enzyme Q, menadiol, 3-hydroxy-1-methylindoline-5,6-diol, 17β-estra-1(10),2,4-triene-3,4,17-triol, reduced 2,6-dichlorophenolindophenol, reduced methyl red, estrone-3,4-quinol, and reduced mytocin C.

In one embodiment of the present invention, the ribosyldihydronicotinamide dehydrogenases (E.C. 1.10.99.2, E.C. 1.10.5.1) or the NAD(P)H dehydrogenases (E.C. 1.6.5.2) catalyze a reaction to convert: a) 1,4-dihydronicotinamide riboside, 1,4-dihydronicotinamide riboside derivatives, or mixtures thereof and b) one or more quinones, into: a) nicotinamide riboside, nicotinamide riboside derivatives, or mixtures thereof, and b) one or more hydroquinones, or vice versa. Non-limiting examples of ribosyldihydronicotinamide dehydrogenases (E.C. 1.10.99.2, E.C. 1.10.5.1) are listed in Table 1.

In another embodiment, the method of making 1,4-dihydronicotinamide riboside, 1,4-dihydronicotinamide riboside derivatives, or mixtures comprises contacting at least the following materials to form a solution:
i. α-D-ribose-1-phosphate, α-D-ribose-1-phosphate derivatives, or mixtures thereof,
ii. nicotinamide, nicotinamide derivatives, or mixtures thereof,
iii. one or more hydroquinones,
iv. one or more pentosyl transferases (E.C. 2.4.2),
v. one or more ribosyldihydronicotinamide dehydrogenases (E.C. 1.10.99.2, E.C. 1.10.5.1), and
vi. one or more solvents;
wherein the solution comprises 1,4-dihydronicotinamide riboside, 1,4-dihydronicotinamide riboside derivatives, or mixtures thereof, one or more quinones, and one or more inorganic orthophosphate anions; further, wherein said one or more inorganic orthophosphate anions are selected from the group consisting of [H₂PO₄]⁻ and [HPO₄]²⁻.

In another embodiment, the method of making 1,4-dihydronicotinamide riboside, 1,4-dihydronicotinamide riboside derivatives, or mixtures thereof comprises contacting at least the following materials to form a solution:
i. 5-phospho-α-D-ribose-1-diphosphate, 5-phospho-α-D-ribose-1-diphosphate derivatives, or mixtures thereof,
ii. nicotinamide, nicotinamide derivatives, or mixtures thereof,
iii. one or more hydroquinones,
iv. one or more pentosyl transferases (E.C. 2.4.2), and
v. one or more 5′-nucleotidases (E.C. 3.1.3.5),
vi. one or more ribosyldihydronicotinamide dehydrogenases (E.C. 1.10.99.2, E.C. 1.10.5.1),
vii. Mg²⁺,
viii. water, and
ix. one or more solvents;
wherein the solution comprises 1,4-dihydronicotinamide riboside, 1,4-dihydronicotinamide riboside derivatives, or mixtures thereof, one or more inorganic diphosphate anions, and one or more inorganic orthophosphate anions, one or more quinones; further, wherein said one or more inorganic diphosphate anions are selected from the group consisting of [HP₂O₇]³⁻, [H₂P₂O₇]²⁻, and [H₃P₂O₇]⁻; and wherein said one or more inorganic orthophosphate anions are selected from the group consisting of [H₂PO₄]⁻ and [HPO₄]²⁻.

### VI. Enzymes

### Enzyme Variants

Enzymes disclosed in the invention are naturally occurring in various organisms. While specific enzymes with the desired activity are used in the examples, the invention is not limited to these enzymes as other enzymes may have similar activities and can be used. For example, nucleoside phosphorylases catalyze the reversible phosphorolysis of purines and pyrimidines. Specifically, purine nucleoside phosphorylase has been demonstrated to catalyze the similar reaction on nicotinamide riboside, although nicotinamide is neither a purine nor a pyrimidine. It may be discovered that some pyrimidine nucleoside phosphorylases may also catalyze this reaction and is disclosed in this invention. Other reactions described in this invention may be catalyzed by enzymes not described in the embodiment, and are also incorporated into the embodiment.

In certain embodiments, variants of these enzymes in which the catalytic activity has been modified, e.g., to make it more active and stable in acidic conditions, may be used in the invention. Amino acid sequence variants of the polypeptide include substitution, insertion, or deletion variants, and variants may be substantially homologous or substantially identical to the unmodified polypeptides. In certain embodiments, the variants retain at least some of the biological activity, e.g., catalytic activity, of the polypeptide. Other variants include variants of the polypeptide that retain at least about 50%, preferably at least about 75%, more preferably at least about 90%, of the biological activity.

Substitutional variants typically exchange one amino acid for another at one or more sites within the protein. Substitutions of this kind can be conservative, that is, one amino acid is replaced with one of similar shape and charge. Conservative substitutions include, for example, the changes of: alanine to serine; arginine to lysine; asparagine to glutamine; aspartate to glutamate; cysteine to serine; glutamine to asparagine; glutamate to aspartate; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. An example of the nomenclature used herein to indicate an amino acid substitution is “S345F ThrA” wherein the naturally occurring serine occurring at position 345 of the naturally occurring ThrA enzyme which has been substituted with a phenylalanine.

A polypeptide or polynucleotide “derived from” an organism contains one or more modifications to the naturally-occurring amino acid sequence or nucleotide sequence and exhibits similar, if not better, activity compared to the native enzyme (e.g., at least 70%, at least 80%, at least 90%, at least 95%, at least 100%, or at least 110% the level of activity of the native enzyme). For example, enzyme activity is improved in some contexts by directed evolution of a parent/naturally-occurring sequence. Additionally or alternatively, an enzyme coding sequence is mutated to achieve feedback resistance.

### Forms of the Enzymes

The isolated enzymes used in this invention are water soluble. It is often preferable to use immobilized enzymes. Immobilized enzymes are often more stable and robust, including examples of purine nucleoside phosphorylases (Journal of Biotechnology (1991) 17: 121-131). Immobilized enzymes are also easier to recover and use in multiple catalytic cycles, thus lowering the cost of an industrial process. Multiple means of enzyme immobilization are known in the art, as summarized in Applied Microbiology and Biotechnology (2015) 99:2065-2082. Enzymes may also be crossed linked to form Cross Linked Enzyme Aggregates (CLEAs; Biotechnology and Bioengineering (2004) 87 (6): 754-762) which are often more stable and are easier to recover and reuse. Many of these enzymes are found together in organisms which can be used as biocatalysts to produce nucleosides (e.g., Journal of Molecular Catalysis B: Enzymatic (2004) 30: 219-227), but they may also be heterologously expressed in engineered microorganisms, which then can be used as biocatalysts. Methods of immobilization and crosslinking of enzymes catalyzing the reactions disclosed in this invention are incorporated herein.

### VII. the Process of Producing Nicotinamide Riboside

### Process Optimization

The reactions disclosed herein can be performed simultaneously or sequentially within one reactor or in multiple reactors. Enzymes and byproducts for a reaction may remain or be removed before the succeeding reaction. The conditions including, but not limited to temperature, pH, solvent, timing of addition of reactants, length of reaction, concentration and agitation may be optimized for each step in the process.

### Production in Engineered Organisms

Enzymes catalyzing some or all of the reactions described in this invention may be expressed in non-natural, engineered heterologous organisms for the production of nicotinamide riboside. Specifically, genes coding for the pathway enzymes may be isolated, inserted into expression vectors used to transform a production organism, may be incorporated into the genome, and direct expression of the enzymes. Protocols used to manipulate organisms are known in the art and explained in publications such as Current Protocols in Molecular Biology, Online ISBN: 9780471142720, John Wiley and Sons, Inc., and Microbial Metabolic Engineering: Methods and Protocols, Qiong Cheng Ed., Springer, and Systems Metabolic Engineering: Methods and Protocols, Hal S. Alper Ed., Springer.

Those familiar to the art can culture the engineered microbial cells to convert feedstocks such as carbohydrates or other precursors into nicotinamide riboside, and recover the nicotinamide riboside. Guidance and protocols can be found in publications such as Fermentation and Biochemical Engineering Handbook: Principles, Process Design, and Equipment, 2^{nd} Edition, Henry C. Vogel and Celeste L. Todaro, Noyes Publications 1997, and Principles of Fermentation Technology, 2^{nd} Edition, P. F. Stanbury et. al., Butterworth Heineman, 2003.

Nicotinamide riboside may be separated from enzymes and reactants, and recovered from the reaction medium using a variety of procedures known in the art. These include, but are not limited to, crystallization, adsorption and release from ionic, hydrophobic and size exclusion resins, filtration, microfiltration, extraction, precipitation as salts or with solvents, or combinations thereof. The extent of the separation required may be limited to the removal of the enzymes, and a mixture of some or all of the remaining products and reactants including nicotinamide riboside, nicotinic acid riboside, 1,4-dihydronicotinamide riboside, nicotinamide, nicotinic acid, 1,4-dihydronicotinamide, inorganic orthophosphate, inorganic diphosphate, α-D-ribose-1-phosphate, 5-phospho-α-D-ribose-1-diphosphate, D-ribose-5-phosphate, β-nicotinamide D-ribonucleotide, and nitrogenous bases may be useful without further purification. Recovery of nicotinamide riboside with or without other products and reactants is a further embodiment of the invention.

### VIII. Examples

### Example 1: Synthesis of Inosine from Inosine 5′-Monophosphate (IMP)

Aliquots of aqueous solutions of inosine 5′-monophosphate disodium salt hydrate (IMP; final concentration 2 mM; C₁₀H₁₁N₄O₈PNa₂.xH₂O; Sigma, St. Louis, Mo.; catalog #14625) and magnesium chloride (final concentration 10 mM; MgCl₂; Sigma, St. Louis, Mo.; catalog #M8266) are mixed in MES buffer (final concentration 50 mM, pH 6.0; C₆H₁₃NO₄S; Sigma, St. Louis, Mo.; catalog # M3671). Then, an aliquot of 5′-nucleotidase (E.C. 3.1.3.5, SEQ ID NO: 3) is added to the solution to initiate the reaction. The solution is incubated at room temperature until significant conversion of IMP is achieved.

### Example 2: Synthesis of α-D-Ribose-1-Phosphate (R1P) from Inosine

Aliquots of D₂O solutions of inosine (final concentration 0.5 mM; C₁₀H₁₂N₄O₅; Sigma, St. Louis, Mo.; catalog #14125), and potassium phosphate (final concentration 0.55 mM; K₃PO₄; Sigma, St. Louis, Mo.; catalog # P5629) were mixed in Tris/D₂O buffer (final concentration 50 mM, pH 7.0; NH₂C(CH₂OH)₃; Sigma, St. Louis, Mo.; catalog # T1503). Then, aliquots of purine nucleoside phosphorylase (final concentration 1.8 mg/mL; E.C. 2.4.2.1, SEQ ID NO: 7) and xanthine oxidase (final concentration 0.7 mg/mL; E.C. 1.17.3.2; SEQ ID NO: 4, 5, and 6) were added to the solution to initiate the reaction. The solution was incubated at room temperature overnight and analyzed by ¹H-NMR and ³¹P-NMR spectroscopy. Full conversion of inosine and formation of R1P was confirmed.

### Example 3: Synthesis of α-D-Ribose-1-Phosphate (R1P) from NR

Aliquots of D₂O solutions of nicotinamide riboside (final concentration 5.0 mM; C₁₁H₁₅N₂O₅Cl; ChromaDex, Irvine, Calif.), and potassium phosphate (final concentration 5.5 mM; K₃PO₄; Sigma, St. Louis, Mo.; catalog # P5629) were mixed in Tris/D₂O buffer (final concentration 50 mM, pH 7.0; NH₂C(CH₂OH)₃; Sigma, St. Louis, Mo.; catalog # T1503). Then, an aliquot of purine nucleoside phosphorylase (final concentration 1.8 mg/mL; E.C. 2.4.2.1, SEQ ID NO: 7) was added to the solution to initiate the reaction. The solution was incubated at room temperature for 2 h and analyzed by ¹H-NMR and ³¹P-NMR spectroscopy. Full conversion of NR to R1P and NAM was confirmed.

### Example 4: Synthesis of NR from α-D-Ribose-1-Phosphate (R1P)

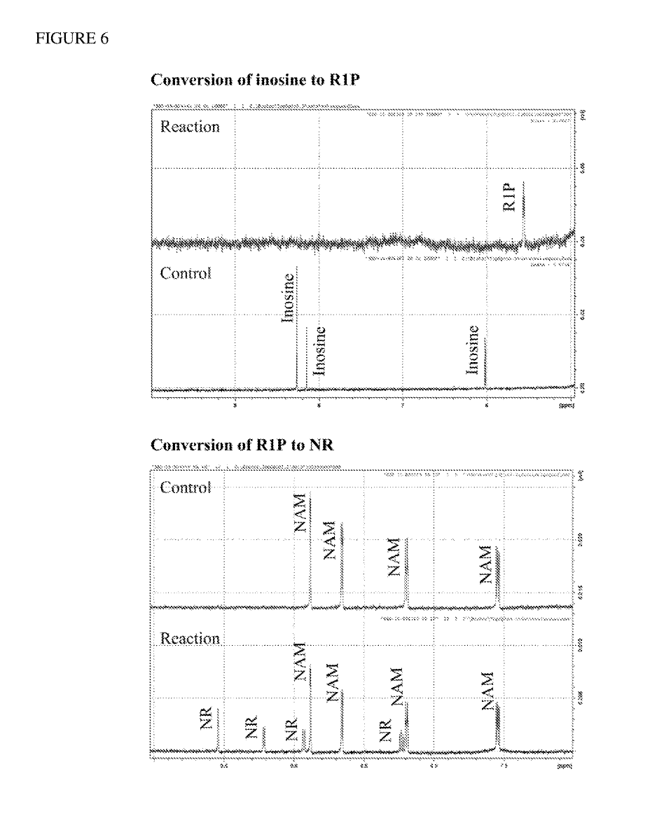

Aliquots of D₂O solutions of α-D-ribose-1-phosphate (R1P, final concentration 2.0 mM; C₅H₁₁O₈P; prepared as described in example 3), nicotinamide (NAM, final concentration 2.0 mM; C₆H₆N₂O; prepared as described in example 3), and sucrose (final concentration 500 mM; C₁₂H₂₂O₁₁; Sigma, St. Louis, Mo.; catalog #84097) were mixed in MES/D₂O buffer (final concentration 100 mM, pH 6.0; C₆H₁₃NO₄S; Sigma, St. Louis, Mo.; catalog # M3671). Then, aliquots of purine nucleoside phosphorylase (final concentration 3.6 mg/mL; E.C. 2.4.2.1, SEQ ID NO: 7) and sucrose phosphorylase (final concentration 0.3 mg/mL; E.C. 2.4.1.7, SEQ ID NO: 8) were added to the solution to initiate the reaction. The solution was incubated at room temperature overnight and analyzed by ¹H-NMR and ³¹P-NMR spectroscopy (FIG. 6). The conversion of NAM into NR was about 30 mol %.

### Example 5: Synthesis of NR from Inosine

Aliquots of aqueous solutions of inosine (final concentration 1.0 mM; C₁₀H₁₂N₄O₅; Sigma, St. Louis, Mo.; catalog #14125), nicotinamide (NAM, final concentration 1.0 mM; C₆H₆N₂O; prepared as described in example 3), potassium phosphate (final concentration 1.05 mM; K₃PO₄; Sigma, St. Louis, Mo.; catalog # P5629), and sucrose (final concentration 500 mM; C₁₂H₂₂O₁₁; Sigma, St. Louis, Mo.; catalog #84097) are mixed in MES/D₂O buffer (final concentration 100 mM, pH 6.0; C₆H₁₃NO₄S; Sigma, St. Louis, Mo.; catalog # M3671). Then, aliquots of purine nucleoside phosphorylase (final concentration 1.8 mg/mL; E.C. 2.4.2.1, SEQ ID NO: 7), xanthine oxidase (final concentration 0.7 mg/mL; E.C. 1.17.3.2; SEQ ID NO: 4, 5, and 6) and sucrose phosphorylase (final concentration 0.3 mg/mL; E.C. 2.4.1.7, SEQ ID NO: 8) are added to the solution to initiate the reaction. The solution is incubated at room temperature until significant conversion of inosine into NR is achieved.

### Example 6: Synthesis of NR from Inosine 5′-Monophosphate (IMP)

Aliquots of aqueous solutions of inosine 5′-monophosphate disodium salt hydrate (IMP; final concentration 2 mM; C₁₀H₁₁N₄O₈PNa₂.xH₂O; Sigma, St. Louis, Mo.; catalog #14625) and magnesium chloride (final concentration 10 mM; MgCl₂; Sigma, St. Louis, Mo.; catalog #M8266) are mixed in MES buffer (final concentration 100 mM, pH 6.0; C₆H₁₃NO₄S; Sigma, St. Louis, Mo.; catalog # M3671). Then, an aliquot of 5′-nucleotidase (E.C. 3.1.3.5, SEQ ID NO: 3), purine nucleoside phosphorylase (E.C. 2.4.2.1, SEQ ID NO: 7), and xanthine oxidase (E.C. 1.17.3.2; SEQ ID NO: 4, 5, and 6) are added to the solution to initiate the reaction. The solution is incubated at room temperature until significant conversion of IMP to R1P is achieved. Then, aliquots of aqueous solutions of nicotinamide (NAM, final concentration 1.0 mM; C₆H₆N₂O; prepared as described in example 3), sucrose (final concentration 500 mM; C₁₂H₂₂O₁₁; Sigma, St. Louis, Mo.; catalog #84097), purine nucleoside phosphorylase (E.C. 2.4.2.1, SEQ ID NO: 7), and sucrose phosphorylase (E.C. 2.4.1.7, SEQ ID NO: 8) are added to the solution, followed by incubation at room temperature until significant conversion of inosine into NR is achieved.

### Example 7: Synthesis of 5-Phospho-α-D-Ribose-1-Diphosphate (PRPP) from Inosine 5′-Monophosphate (IMP)

Aliquots of aqueous solutions of inosine 5′-monophosphate disodium salt hydrate (IMP; final concentration 2 mM; C₁₀H₁₁N₄O₈PNa₂.xH₂O; Sigma, St. Louis, Mo.; catalog #14625), sodium pyrophosphate tetrabasic (PP, final concentration 2 mM; Na₄P₂O₇; Aldrich, St. Louis, Mo.; catalog # P8010), and magnesium chloride (final concentration 10 mM; MgCl₂; Sigma, St. Louis, Mo.; catalog # M8266) are mixed in MES buffer (final concentration 50 mM, pH 6.0; C₆H₁₃NO₄S; Sigma, St. Louis, Mo.; catalog # M3671). Then, aliquots of hypoxanthine phosphoribosyl transferase (E.C. 2.4.2.7, SEQ ID NO: 9) and xanthine oxidase (E.C. 1.17.3.2; SEQ ID NO: 4, 5, and 6) are added to the solution to initiate the reaction. The solution is incubated at room temperature until significant conversion of IMP is achieved.

### Example 8: Synthesis of β-Nicotinamide Mononucleotide (MNM) from 5-Phospho-α-D-Ribose-1-Diphosphate (PRPP)

Aliquots of aqueous solutions of nicotinamide (NAM, final concentration 1 mM; C₆H₆N₂O; Sigma, St. Louis, Mo.; catalog #72340), 5-phospho-α-D-ribose-1-diphosphate pentasodium salt (PRPP, final concentration 1 mM; C₅H₈Na₅O₁₄P₃; Sigma, St. Louis, Mo.; catalog # P8296), and magnesium chloride (final concentration 10 mM; MgCl₂; Sigma, St. Louis, Mo.; catalog # M8266) are mixed in MES buffer (final concentration 50 mM, pH 6.0; C₆H₁₃NO₄S; Sigma, St. Louis, Mo.; catalog # M3671). Then, aliquots of nicotinamide phosphoribosyl transferase (E.C. 2.4.2.12, SEQ ID NO: 10) and inorganic diphosphatase (E.C. 3.6.1.1, SEQ ID NO: 11) are added to the solution to initiate the reaction. The solution is incubated at room temperature until significant conversion of PRPP is achieved.

### Example 9: Synthesis of 3-Nicotinamide Mononucleotide (MNM) from 5-Phospho-α-D-Ribose-1-Diphosphate (PRPP)

Aliquots of aqueous solutions of nicotinamide (NAM, final concentration 1 mM; C₆H₆N₂O; Sigma, St. Louis, Mo.; catalog #72340), 5-phospho-α-D-ribose-1-diphosphate pentasodium salt (PRPP, final concentration 1 mM; C₅H₈Na₅O₄P₃; Sigma, St. Louis, Mo.; catalog # P8296), magnesium chloride (final concentration 10 mM; MgCl₂; Sigma, St. Louis, Mo.; catalog # M8266), and iron(III) chloride (final concentration 100 mM; FeCl₃; Sigma-Aldrich, St. Louis, Mo.; catalog #157740) are mixed in MES buffer (final concentration 50 mM, pH 6.0; C₆H₁₃NO₄S; Sigma, St. Louis, Mo.; catalog # M3671). Then, aliquots of nicotinamide phosphoribosyl transferase (E.C. 2.4.2.12, SEQ ID NO: 10) and inorganic diphosphatase (E.C. 3.6.1.1, SEQ ID NO: 11) are added to the solution to initiate the reaction. The solution is incubated at room temperature until significant conversion of PRPP is achieved.

### Example 10: Synthesis of NR from β-Nicotinamide Mononucleotide (NMN)

Aliquots of β-nicotinamide mononucleotide (NMN, final concentration 1 mM; C₁₁H₁₅N₂O₈P; Sigma, St. Louis, Mo.; catalog # N3501), and magnesium chloride (final concentration 10 mM; MgCl₂; Sigma, St. Louis, Mo.; catalog # M8266) are suspended in MES buffer (final concentration 50 mM, pH 6.0; C₆H₁₃NO₄S; Sigma, St. Louis, Mo.; catalog #M3671). Then, an aliquot of 5′-nucleotidase (E.C. 3.1.3.5, SEQ ID NO: 3) is added to the solution to initiate the reaction. The solution is incubated at room temperature until significant conversion of NMN is achieved.

### Example 11: Synthesis of NR from 5-Phospho-α-D-Ribose-1-Diphosphate (PRPP)

Aliquots of aqueous solutions of nicotinamide (NAM, final concentration 1 mM; C₆H₆N₂O; Sigma, St. Louis, Mo.; catalog #72340), 5-phospho-α-D-ribose-1-diphosphate pentasodium salt (PRPP, final concentration 1 mM; C₅H₈Na₅O₁₄P₃; Sigma, St. Louis, Mo.; catalog # P8296), and magnesium chloride (final concentration 10 mM; MgCl₂; Sigma, St. Louis, Mo.; catalog # M8266) are mixed in MES buffer (final concentration 50 mM, pH 6.0; C₆H₁₃NO₄S; Sigma, St. Louis, Mo.; catalog # M3671). Then, aliquots of nicotinamide phosphoribosyl transferase (E.C. 2.4.2.12, SEQ ID NO: 10) and 5′-nucleotidase (E.C. 3.1.3.5, SEQ ID NO: 3) are added to the solution to initiate the reaction. The solution is incubated at room temperature until significant conversion of PRPP is achieved.

### Example 12: Synthesis of NR from 5-Phospho-α-D-Ribose-1-Diphosphate (PRPP)

Aliquots of aqueous solutions of nicotinamide (NAM, final concentration 1 mM; C₆H₆N₂O; Sigma, St. Louis, Mo.; catalog #72340), 5-phospho-α-D-ribose-1-diphosphate pentasodium salt (PRPP, final concentration 1 mM; C₅H₈Na₅O₁₄P₃; Sigma, St. Louis, Mo.; catalog # P8296), and magnesium chloride (final concentration 10 mM; MgCl₂; Sigma, St. Louis, Mo.; catalog # M8266) are mixed in MES buffer (final concentration 50 mM, pH 6.0; C₆H₁₃NO₄S; Sigma, St. Louis, Mo.; catalog # M3671). Then, aliquots of nicotinamide phosphoribosyl transferase (E.C. 2.4.2.12, SEQ ID NO: 10), 5′-nucleotidase (E.C. 3.1.3.5, SEQ ID NO: 3), and inorganic diphosphatase (E.C. 3.6.1.1, SEQ ID NO: 11) are added to the solution to initiate the reaction. The solution is incubated at room temperature until significant conversion of PRPP is achieved.

### Example 13: Synthesis of NR from 5-Phospho-α-D-Ribose-1-Diphosphate (PRPP)

Aliquots of aqueous solutions of nicotinamide (NAM, final concentration 1 mM; C₆H₆N₂O; Sigma, St. Louis, Mo.; catalog #72340), 5-phospho-α-D-ribose-1-diphosphate pentasodium salt (PRPP, final concentration 1 mM; C₅H₈Na₅O₁₄P₃; Sigma, St. Louis, Mo.; catalog # P8296), magnesium chloride (final concentration 10 mM; MgCl₂; Sigma, St. Louis, Mo.; catalog # M8266), and iron(III) chloride (final concentration 100 mM; FeCl₃; Sigma-Aldrich, St. Louis, Mo.; catalog #157740) are mixed in MES buffer (final concentration 50 mM, pH 6.0; C₆H₁₃NO₄S; Sigma, St. Louis, Mo.; catalog # M3671). Then, aliquots of nicotinamide phosphoribosyl transferase (E.C. 2.4.2.12, SEQ ID NO: 10) and 5′-nucleotidase (E.C. 3.1.3.5, SEQ ID NO: 3) are added to the solution to initiate the reaction. The solution is incubated at room temperature until significant conversion of PRPP is achieved.

### Example 14: Synthesis of NR from Inosine 5′-Monophosphate (IMP)

Aliquots of aqueous solutions of inosine 5′-monophosphate disodium salt hydrate (IMP; final concentration 2 mM; C₁₀H₁₁N₄O₈PNa₂.xH₂O; Sigma, St. Louis, Mo.; catalog #14625), sodium pyrophosphate tetrabasic (PP, final concentration 2 mM; Na₄P₂O₇; Aldrich, St. Louis, Mo.; catalog # P8010), and magnesium chloride (final concentration 20 mM; MgCl₂; Sigma, St. Louis, Mo.; catalog # M8266) are mixed in MES buffer (final concentration 100 mM, pH 6.0; C₆H₁₃NO₄S; Sigma, St. Louis, Mo.; catalog # M3671). Then, aliquots of hypoxanthine phosphoribosyl transferase (E.C. 2.4.2.7, SEQ ID NO: 9) and xanthine oxidase (E.C. 1.17.3.2; SEQ ID NO: 4, 5, and 6) are added to the solution to initiate the reaction. The solution is incubated at room temperature until significant conversion of IMP to PRPP is achieved.

Then, aliquots of aqueous solutions of nicotinamide (NAM, final concentration 1 mM; C₆H₆N₂O; Sigma, St. Louis, Mo.; catalog #72340), nicotinamide phosphoribosyl transferase (E.C. 2.4.2.12, SEQ ID NO: 10), and 5′-nucleotidase (E.C. 3.1.3.5, SEQ ID NO: 3) are added to the solution. The solution is incubated at room temperature until significant conversion of PRPP to nicotinamide riboside is achieved.

### Example 15: Synthesis of NR from Inosine 5′-Monophosphate (IMP)

Aliquots of aqueous solutions of inosine 5′-monophosphate disodium salt hydrate (IMP; final concentration 2 mM; C₁₀H₁₁N₄O₈PNa₂.xH₂O; Sigma, St. Louis, Mo.; catalog #14625), sodium pyrophosphate tetrabasic (PP, final concentration 2 mM; Na₄P₂O₇; Aldrich, St. Louis, Mo.; catalog # P8010), and magnesium chloride (final concentration 20 mM; MgCl₂; Sigma, St. Louis, Mo.; catalog # M8266) are mixed in MES buffer (final concentration 100 mM, pH 6.0; C₆H₁₃NO₄S; Sigma, St. Louis, Mo.; catalog # M3671). Then, aliquots of hypoxanthine phosphoribosyl transferase (E.C. 2.4.2.7, SEQ ID NO: 9) and xanthine oxidase (E.C. 1.17.3.2; SEQ ID NO: 4, 5, and 6) are added to the solution to initiate the reaction. The solution is incubated at room temperature until significant conversion of IMP to PRPP is achieved.

Then, aliquots of aqueous solutions of nicotinamide (NAM, final concentration 1 mM; C₆H₆N₂O; Sigma, St. Louis, Mo.; catalog #72340), nicotinamide phosphoribosyl transferase (E.C. 2.4.2.12, SEQ ID NO: 10), 5′-nucleotidase (E.C. 3.1.3.5, SEQ ID NO: 3), and inorganic diphosphatase (E.C. 3.6.1.1, SEQ ID NO: 11) are added to the solution. The solution is incubated at room temperature until significant conversion of PRPP to nicotinamide riboside is achieved.

### Example 16: Synthesis of NR from Inosine 5′-Monophosphate (IMP)

Aliquots of aqueous solutions of inosine 5′-monophosphate disodium salt hydrate (IMP; final concentration 2 mM; C₁₀H₁₁N₄O₈PNa₂.xH₂O; Sigma, St. Louis, Mo.; catalog #14625), sodium pyrophosphate tetrabasic (PP, final concentration 2 mM; Na₄P₂O₇; Aldrich, St. Louis, Mo.; catalog # P8010), and magnesium chloride (final concentration 20 mM; MgCl₂; Sigma, St. Louis, Mo.; catalog # M8266) are mixed in MES buffer (final concentration 100 mM, pH 6.0; C₆H₁₃NO₄S; Sigma, St. Louis, Mo.; catalog # M3671). Then, aliquots of hypoxanthine phosphoribosyl transferase (E.C. 2.4.2.7, SEQ ID NO: 9) and xanthine oxidase (E.C. 1.17.3.2; SEQ ID NO: 4, 5, and 6) are added to the solution to initiate the reaction. The solution is incubated at room temperature until significant conversion of IMP to PRPP is achieved.

Then, aliquots of aqueous solutions of nicotinamide (NAM, final concentration 1 mM; C₆H₆N₂O; Sigma, St. Louis, Mo.; catalog #72340), iron(III) chloride (final concentration 100 mM; FeCl₃; Sigma-Aldrich, St. Louis, Mo.; catalog #157740), nicotinamide phosphoribosyl transferase (E.C. 2.4.2.12, SEQ ID NO: 10), and 5′-nucleotidase (E.C. 3.1.3.5, SEQ ID NO: 3) are added to the solution. The solution is incubated at room temperature until significant conversion of PRPP to nicotinamide riboside is achieved.

### IX. Tables

TABLE 1
List of exemplary enzymes included in the current invention.

| E.C. | Enzyme Name | Organism | UniProt |
|---|---|---|---|
| 1.10.5.1 | Ribosyldihydronicotinamide dehydrogenase | Homo sapiens | P16083 |
| 1.10.5.1 | Ribosyldihydronicotinamide dehydrogenase | Mus musculus | Q9JI75 |
| 1.17.3.2 | Xanthine oxidoreductase | Blastobotrys adeninivorans | R4ZGN4 |
| 1.17.3.2 | Xanthine dehydrogenase | Escherichia coli K12 | Q46799, |
| | | | Q46800, |
| | | | Q46801 |
| 1.17.3.2 | Xanthine dehydrogenase | Homo sapiens | P47989 |
| 1.17.3.2 | Xanthine oxidase | Mus musculus | Q00519 |
| 1.17.3.2 | Xanthine oxidoreductase | Rattus norvegicus | P22985 |
| 2.4.1.7 | Sucrose phosphorylase | Bifidobacterium adolescentis | Q84HQ2 |
| 2.4.1.7 | Sucrose phosphorylase | Leuconostoc mesenteroides | Q59495 |
| 2.4.1.7 | Sucrose phosphorylase | Streptococcus mutans | P10249 |
| 2.4.2.1 | S-methyl-5-thioadenosine phosphorylase | Aeropyrum pemix | Q9YDC0 |
| 2.4.2.1 | Purine nucleoside phosphorylase | Bacillus halodurans | Q9KCN8 |
| 2.4.2.1 | Purine nucleoside phosphorylase | Bos taurus | P55859 |
| 2.4.2.1 | Purine nucleoside phosphorylase | Cellulomonas sp. | P81989 |
| 2.4.2.1 | Xanthine phosphorylase | Escherichia coli K-12 | P45563 |
| | | substr. MG1655 | |
| 2.4.2.1 | Purine nucleoside phosphorylase | Geobacillus | P77834 |
| | | stearothermophilus | |
| 2.4.2.1 | Purine nucleoside phosphorylase | Homo sapiens | P00491 |
| 2.4.2.1 | Purine nucleoside phosphorylase | Pyrococcus furiosus | Q8U2I1 |
| 2.4.2.1 | Purine nucleoside phosphorylase | Thermus thermophilus | Q72IR2 |
| 2.4.2.10 | UMP synthase | Arabidopsis thaliana col | Q42586 |
| 2.4.2.10 | Orotate phosphoribosyltransferase | Escherichia coli K-12 | P0A7E3 |
| | | substr. MG1655 | |
| 2.4.2.12 | Nicotinamide phosphoribosyltransferase | Haemophilus ducreyi | Q9APM3 |
| 2.4.2.12 | Nicotinamide phosphoribosyltransferase | Homo sapiens | P43490 |
| 2.4.2.12 | Nicotinamide phosphoribosyltransferase | Mus musculus | Q99KQ4 |
| 2.4.2.12 | Nicotinamide phosphoribosyltransferase | Rattus norvegicus | Q80Z29 |
| 2.4.2.12 | Nicotinamide phosphoribosyltransferase | Sus scrofa | Q52I78 |
| 2.4.2.12 | Nicotinamide phosphoribosyltransferase | Synechocystis sp. PCC 6803 | Q55929 |
| 2.4.2.2 | Pyrimidine nucleoside phosphorylase | Bacillus subtilis | P39142 |
| 2.4.2.2 | Pyrimidine nucleoside phosphorylase | Geobacillus | P77836 |
| | | stearothermophilus | |
| 2.4.2.2 | Pyrimidine nucleoside phosphorylase | Thermus thermophilus | Q72HS4 |
| 2.4.2.22 | Xanthine-guanine phosphoribosyltransferase | Escherichia coli K-12 | P0A9M5 |
| | | substr. MG1655 | |
| 2.4.2.3 | Uridine phosphorylase | Aeropyrum pernix | Q9YA34 |
| 2.4.2.3 | Uridine phosphorylase | Escherichia coli K-12 | P12758 |
| | | substr. MG1655 | |
| 2.4.2.3 | Uridine phosphorylase | Haemophilus influenzae | P43770 |
| 2.4.2.3 | Uridine phosphorylase | Homo sapiens | Q16831 |
| 2.4.2.7 | Adenine phosphoribosyltransferase | Arabidopsis thaliana col | P31166 |
| 2.4.2.7 | Adenine phosphoribosyltransferase | Escherichia coli K-12 | P69503 |
| | | substr. MG1655 | |
| 2.4.2.7 | Adenine phosphoribosyltransferase | Saccharomyces cerevisiae | P49435 |
| 2.4.2.8 | Hypoxanthine-guanine | Arabidopsis thaliana col | Q8L8L7 |
| | phosphoribosyltransferase | | |
| 2.4.2.8 | Hypoxanthine-guanine | Cricetulus griseus | P00494 |
| | phosphoribosyltransferase | | |
| 2.4.2.8 | Hypoxanthine phosphoribosyltransferase | Escherichia coli K-12 | P0A9M2 |
| | | substr. MG1655 | |
| 2.4.2.8 | Hypoxanthine-guanine | Halobacterium salinarum | Q9HRT1 |
| | phosphoribosyltransferase | | |
| 2.4.2.8 | Hypoxanthine-guanine | Homo sapiens | P00492 |
| | phosphoribosyltransferase | | |
| 2.4.2.8 | Hypoxanthine-guanine | Saccharomyces cerevisiae | Q04178 |
| | phosphoribosyltransferase | | |
| 2.4.2.8 | Hypoxanthine-guanine | Trypanosoma brucei brucei | Q07010 |
| | phosphoribosyltransferase | | |
| 2.4.2.9 | Uracil phosphoribosyltransferase | Arabidopsis thaliana col | Q9M336 |
| 2.4.2.9 | Uracil phosphoribosyltransferase | Escherichia coli K-12 | P0A8F0 |
| | | substr. MG1655 | |
| 2.4.2.9 | Uracil phosphoribosyltransferase | Saccharomyces cerevisiae | P18562 |
| 3.1.3.1 | Alkaline phosphatase | Escherichia coli K-12 | P00634 |
| | | substr. MG1655 | |
| 3.1.3.1 | Farnesyl diphosphatase | Saccharomyces cerevisiae | P11491 |
| 3.1.3.2 | Acid phosphatase | Escherichia coli K-12 | P0AE22 |
| | | substr. MG1655 | |
| 3.1.3.2 | Acid phosphatase | Nicotiana tabacum | Q84KS8 |
| 3.1.3.5 | 5′-Nucleotidase | Arachis hypogaea | B4UWA8 |
| 3.1.3.5 | 5′-Nucleotidase, NAD pyrophosphatase | Haemophilus influenzae | P44569 |
| 3.1.3.5 | 5′-Nucleotidase, NMN 5′-nucleotidase | Haemophilus influenzae | P26093 |
| 3.1.3.5 | 5′-Nucleotidase | Homo sapiens | P21589 |
| 3.2.2.1 | Purine nucleosidase | Bacillus thuringiensis | A7UHH1 |
| 3.2.2.1 | Purine nucleosidase | Lupinus luteus | B6DX57 |
| 3.2.2.1 | Purine nucleosidase | Sulfolobus solfataricus | Q97WH6 |
| 3.2.2.1 | Purine nucleosidase | Trypanosoma vivax | Q9GPQ4 |
| 3.2.2.10 | Pyrimidine-5′-nucleotide nucleosidase | Alcaligenes faecalis | |
| 3.2.2.10 | Pyrimidine-5′-nucleotide nucleosidase | Homo sapiens | |
| 3.2.2.10 | Pyrimidine-5′-nucleotide nucleosidase | Neisseria meningitidis | |
| 3.2.2.10 | Pyrimidine-5′-nucleotide nucleosidase | Streptomyces virginiae | |
| 3.2.2.12 | Inosinate nucleosidase | Aspergillus oryzae | |
| 3.2.2.14 | NMN nucleosidase | Azotobacter vinelandii | |
| 3.2.2.14 | NMN nucleosidase | Escherichia coli | |
| 3.2.2.14 | NMN nucleosidase | Haemophilus influenzae | |
| 3.2.2.14 | NMN nucleosidase | Nicotiana tabacum | |
| 3.2.2.14 | NMN nucleosidase | Salmonella enterica subsp. | |
| | | enterica serovar | |
| | | Typhimurium | |
| 3.6.1.1 | Inorganic diphosphatase | Aquifex aeolicus | O67501 |
| 3.6.1.1 | Inorganic diphosphatase | Escherichia coli K-12 | P0A7A9 |
| | | substr. MG1655 | |
| 3.6.1.1 | Inorganic diphosphatase | Helicobacter pylori | P56153 |
| 3.6.1.1 | Inorganic diphosphatase | Mycoplasma pneumoniae | P75250 |
| | | M129 | |
| 3.6.1.1 | Inorganic diphosphatase | Pyrococcus horikoshii | O59570 |
| 3.6.1.1 | Inorganic diphosphatase | Sulfolobus acidocaldarius | P50308 |
| 5.4.2.7 | Phosphopentomutase | Escherichia coli K-12 | P0A6K6 |
| | | substr. MG1655 | |
| 5.4.2.7 | Phosphoglucomutase | Homo sapiens | Q96G03 |
| 6.3.4.21 | Nicotinate phosphoribosyltransferase | Escherichia coli K12 | P18133 |
| 6.3.4.21 | Nicotinate phosphoribosyltransferase | Homo sapiens | Q6XQN6 |
| 6.3.4.21 | Nicotinate phosphoribosyltransferase | Mus musculus | Q8CC86 |
| 6.3.4.21 | Nicotinate phosphoribosyltransferase | Mycobacterium tuberculosis | P9WJW5 |
| 6.3.4.21 | Nicotinate phosphoribosyltransferase | Rattus norvegicus | Q6XQN1 |
| 6.3.4.21 | Nicotinate phosphoribosyltransferase | Thermoplasma acidophilum | Q9HJ28 |
| 3.5.1.42 | Nicotinamide-nucleotide amidohydrolase | Escherichia coli K12 | P0A6G3 |
| 3.5.1.42 | Nicotinamide-nucleotide amidohydrolase | Shewanella oneidensis | Q8EK32 |
| | | MR-1 | |

The foregoing description is given for clearness of understanding only, and no unnecessary limitations should be understood therefrom, as modifications within the scope of the invention may be apparent to those having ordinary skill in the art.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method of making nicotinamide riboside, nicotinamide riboside derivatives, or mixtures thereof comprising the steps of:
c. contacting at least the following materials to form a solution:
i. 5-phospho-α-D-ribose-1-diphosphate, 5-phospho-α-D-ribose-1-diphosphate derivatives, or mixtures thereof,
ii. nicotinamide, nicotinamide derivatives, or mixtures thereof,
iii. one or more pentosyl transferases (E.C. 2.4.2),
iv. Mg²⁺, and
v. one or more solvents;
wherein the solution comprises β-nicotinamide D-ribonucleotide, β-nicotinamide D-ribonucleotide derivatives, or mixtures thereof and one or more inorganic diphosphate anions; further, wherein said one or more inorganic diphosphate anions are selected from the group consisting of [HP₂O₇]³⁻, [H₂P₂O₇]²⁻, and [H₃P₂O₇]⁻; and
d. Contacting said β-nicotinamide D-ribonucleotide, β-nicotinamide D-ribonucleotide derivatives, or mixtures thereof with at least the following materials to form a solution:
i. one or more phosphoric monoester hydrolases (E.C. 3.1.3),
ii. water, and
iii. one or more solvents;
wherein the solution comprises nicotinamide riboside, nicotinamide riboside derivatives, or mixtures thereof and one or more inorganic orthophosphate anions; wherein said one or more inorganic orthophosphate anions are selected from the group consisting of [H₂PO₄]⁻ and [HPO₄]²⁻.

2. The method of claim 1, wherein said one or more pentosyl transferases are selected from the group consisting of nicotinamide phosphoribosyl transferases (E.C. 2.4.2.12).

3. The method of claim 1, wherein said one or more phosphoric monoester hydrolases (E.C. 3.1.3) are selected from the group consisting of alkaline phosphatases (E.C. 3.1.3.1), acid phosphatases (E.C. 3.1.3.2), 5′-nucleotidases (E.C. 3.1.3.5), and mixtures thereof.

4. The method of claim 1, wherein said one or more phosphoric monoester hydrolases (E.C. 3.1.3) are selected from the group consisting of 5′-nucleotidases (E.C. 3.1.3.5).

5. The method of claim 1, further comprising contacting said one or more inorganic diphosphate anions with at least one or more inorganic diphosphatases (E.C. 3.6.1.1) and water to remove said one or more inorganic diphosphate anions from said solution.

6. The method of claim 1, further comprising contacting said one or more inorganic diphosphate anions with one or more cations to produce one or more diphosphate salts; wherein said one or more diphosphate salts are essentially insoluble in said one or more solvents.

7. The method of claim 6, wherein said one or more cations are selected from the group consisting of Li⁺, Mg²⁺, Ca²⁺, Sr²⁺, Ba²⁺, Mn²⁺, Fe³⁺, Co²⁺, Ni²⁺, Cu²⁺, Ag⁺, Zn²⁺, Cd²⁺, Al³⁺, Pb²⁺, Bi³⁺, and mixtures thereof; and wherein said one or more solvents is water.

8. The method of claim 6, wherein said one or more cations are selected from the group consisting of Mg²⁺, Ca²⁺, Ba²⁺, Fe³⁺, Al³⁺, and mixtures thereof; and further, wherein said one or more solvents is water.

9. The method of claim 1, further comprising contacting said one or more inorganic diphosphate anions with an adsorbent material to remove said one or more inorganic diphosphate anions from said solution.

10. The method of claim 41, further comprising removing said one or more inorganic orthophosphate anions from said solution comprising nicotinamide riboside, nicotinamide riboside derivatives, or mixtures thereof.

11. The method of claim 10, wherein said one or more inorganic orthophosphate anions are removed by a method comprising contacting said one or more inorganic orthophosphate anions with at least the following materials:
a. one or more phosphate acceptors, and
b. one or more phosphorylases;
to produce a phosphate ester.

12. The method of claim 11, wherein said one or more phosphate acceptors is selected from the group consisting of polysaccharides and disaccharides.

13. The method of claim 11, wherein said one or more phosphate acceptors is sucrose.

14. The method of claim 11, wherein said one or more phosphorylases are selected from the group consisting of glycogen phosphorylases (E.C. 2.4.1.1), sucrose phosphorylases (E.C. 2.4.1.7), maltose phosphorylases (E.C. 2.4.1.8), cellobiose phosphorylases (E.C. 2.4.1.20), 1,3-β-oligoglucan phosphorylases (E.C. 2.4.1.30), laminaribiose phosphorylases (E.C. 2.4.1.31), cellodextrin phosphorylases (E.C. 2.4.1.49), α,α-trehalose phosphorylases (E.C. 2.4.1.64 and E.C. 2.4.1.231), 1,3-β-D-glucan phosphorylases (E.C. 2.4.1.97), 1,3-β-galactosyl-N-acetylhexosamine phosphorylases (E.C. 2.4.1.216), trehalose 6-phosphate phosphorylases (E.C. 2.4.1.216), kojibiose phosphorylases (E.C. 2.4.1.230), β-D-galactosyl-(1-4)-L-rhamnose phosphorylases (E.C. 2.4.1.247), nigerose phosphorylases (E.C. 2.4.1.279), N,N′-diacetylchitobiose phosphorylases (E.C. 2.4.1.280), 4-O-β-D-mannosyl-D-glucose phosphorylases (E.C. 2.4.1.281), 3-O-α-D-glucosyl-L-rhamnose phosphorylase (E.C. 2.4.1.282), and mixtures thereof.

15. The method of claim 11, wherein said one or more phosphorylases are selected from the group consisting of sucrose phosphorylases (E.C. 2.4.1.7).

16. The method of claim 10, wherein said one or more inorganic orthophosphate anions are removed by a method comprising contacting said one or more inorganic orthophosphate anions with one or more cations to produce one or more phosphate salts; wherein said one or more phosphate salts are essentially insoluble in said one or more solvents.

17. The method of claim 16, wherein said one or more cations are selected from the group consisting of Li⁺, Mg²⁺, Ca²⁺, Sr²⁺, Ba²⁺, Mn²⁺, Fe³⁺, Co²⁺, Ni²⁺, Cu²⁺, Ag⁺, Zn²⁺, Cd²⁺, Al³⁺, Pb²⁺, Bi³⁺, and mixtures thereof; and further, wherein said one or more solvents is water.

18. The method of claim 16, wherein said one or more cations are selected from the group consisting of Mg²⁺, Ca²⁺, Ba²⁺, Fe³⁺, Al³⁺, and mixtures thereof; and further, wherein said one or more solvents is water.

19. The method of claim 10, wherein said one or more inorganic orthophosphate anions are removed by a method comprising adsorbing said one or more inorganic orthophosphate anions onto an adsorbent.

20. The method of claim 1, where said one or more solvents are selected from the group consisting of water, alcohols, esters, ethers, ketones, nitriles, amides, sulfoxides, hydrocarbons, chlorinated hydrocarbons, and mixtures thereof.

21. The method of claim 1, where said one or more solvents are selected from the group consisting of water, methanol, ethanol, n-propanol, iso-propanol, n-butanol, iso-butanol, sec-butanol, tert-butanol, ethyl acetate, tetrahydrofuran, acetone, methyl ethyl ketone, methyl n-propyl ketone, methyl n-butyl ketone, acetonitrile, dimethylformamide, dimethyl sulfoxide, and mixtures thereof.

22. The method of claim 1, wherein said one or more inorganic diphosphate anions and said one or more inorganic orthophosphate anions are essentially insoluble in said one or more solvents.

23. The method of claim 1, wherein said 5-phospho-α-D-ribose-1-diphosphate, 5-phospho-α-D-ribose-1-diphosphate derivatives, or mixtures thereof are produced by a method comprising contacting at least the following materials:
a. one or more ribonucleotides,
b. one or more inorganic diphosphate anions,
c. Mg²⁺,
d. one or more pentosyl transferases (E.C. 2.4.2), and
e. one or more solvents.

24. The method of claim 23, wherein said one or more ribonucleotides are selected from the group consisting of purine ribonucleotides and mixtures thereof.

25. The method of claim 23, wherein said one or more ribonucleotides are selected from the group consisting of inosine 5′-monophosphate, guanosine 5′-monophosphate, and mixtures thereof.

26. The method of claim 23, wherein said one or more ribonucleotides is inosine 5′-monophosphate.

27. The method of claim 26, further comprising contacting said inosine 5′-monophosphate, said one or more inorganic diphosphate anions, said one or more pentosyl transferases (E.C. 2.4.2), and said one or more solvents with at least one or more xanthine oxidases (E.C. 1.17.3.2) and oxygen; and wherein said one or more solvents comprise water.

28. The method of claim 23, wherein the contacting of said one or more ribonucleotides, said one or more inorganic diphosphate anions, said one or more pentosyl transferases (E.C. 2.4.2), and said one or more solvents produces one or more essentially insoluble free nitrogenous bases in said one or more solvents.

29. The method of claim 23, wherein said one or more pentosyl transferases (E.C. 2.4.2) are selected from the group consisting of adenine phosphoribosyl transferases (E.C. 2.4.2.7), hypoxanthine phosphoribosyl transferases (E.C. 2.4.2.8), uracil phosphoribosyl transferases (2.4.2.9), orotate phosphoribosyl transferase (E.C. 2.4.2.10), amidophosphoribosyl transferase (E.C. 2.4.2.14), anthranilate phosphoribosyl transferase (2.4.2.18), dioxotetrahydropyrimidine phosphoribosyl transferase (2.4.2.20), xanthine phosphoribosyl transferase (2.4.2.22), and mixtures thereof.

30. The method of claim 23, wherein said one or more ribonucleotides is inosine 5′-monophosphate and wherein said one or more pentosyl transferases are hypoxanthine phosphoribosyl transferases (E.C. 2.4.2.8).

31. The method of claim 1, wherein said nicotinamide, nicotinamide derivatives, or mixtures thereof comprises nicotinic acid and wherein said nicotinamide riboside, nicotinamide riboside derivatives, or mixtures thereof comprises nicotinic acid riboside.

32. The method of claim 31, wherein said nicotinic acid riboside is further contacted with an ammonium source and an amidase (E.C. 3.5.1).

33. The method of claim 32, wherein said ammonium source is selected from the group consisting of ammonia, amino acids, and mixtures thereof.

34. The method of claim 1, wherein said one or more solvents comprise water and wherein the pH of said solution is between about 1 and about 10.

35. The method of claim 1, wherein said one or more solvents comprise water and wherein the pH of said solution is between about 2 and about 7.

36. The method of claim 1, wherein said one or more solvents comprise water and wherein the pH of said solution is between about 3 and about 5.


SEQUENCE LISTING (12 sequences)

SEQ ID NO: 1 - protein, 244 aa, Aeropyrum pernix
MRKPVHLEAGPGDVAPLVVAVGDPGRAERLATGLLEDARLVSSARGLKVYTGSFNGSEVTIATHGIGGPSAAVVFEELRMLGAEVLVRLGTSGGLSKDLRLGDVVVAAGAGCYWGSGGSIQYAGERPMCLPASPDPILTAGIYRGLSSRLGDRVVLAPVMSSDAFYAETPEAAGRWRSLGMAAVEMELHTLFSISWIRGFRSAGVLIVSDLLLPEGFKRITPGELARREVEVGRALLEVLTGGV

SEQ ID NO: 2 - protein, 481 aa, Streptococcus mutans
MPIINKTMLITYADSLGKNLKELNENIENYFGDAVGGVHLLPFFPSTGDRGFAPIDYHEVDSAFGDWDDVKCLGEKYYLMFDFMINHISRQSKYYKDYQEKHEASAYKDLFLNWDKFWPKNRPTQEDVDLIYKRKDRAPKQEIQFADGSVEHLWNTFGEEQIDLDVTKEVTMDFIRSTIENLAANGCDLIRLDAFAYAVKKLDTNDFFVEPEIWTLLDKVRDIAAVSGAEILPEIHEHYTIQFKIADHDYYVYDFALPMVTLYSLYSSKVDRLAKWLKMSPMKQFTTLDTHDGIGVVDVKDILTDEEITYTSNELYKVGANVNRKYSTAEYNNLDIYQINSTYYSALGDDDQKYFLARLIQAFAPGIPQVYYVGFLAGKNDLELLESTKEGRNINRHYYSSEEIAKEVKRPVVKALLNLFTYRNQSAAFDLDGRIEVETPNEATIVIERQNKDGSHIAKAEINLQDMTYRVTENDQTISFE

SEQ ID NO: 3 - protein, 578 aa, Haemophilus influenzae
KEAPQAHKAVELSILHINDHHSYLEPHETRINLNGQQTKVDIGGFSAVNAKLNKLRKKYKNPLVLHAGDAITGTLYFTLFGGSADAAVMNAGNFHYFTLGNHEFDAGNEGLLKLLEPLKIPVLSANVIPDKNSILYNKWKPYDIFTVDGEKIAIIGLDTVNKTVNSSSPGKDVKFYDEIATAQIMANALKQQGINKIILLSHAGSEKNIEIAQKVNDIDVIVTGDSHYLYGNDELRSLKLPVIYEYPLEFKNPNGDPVFVMEGWAYSAVVGDLGVKFSPEGIASITRKIPHVLMSSHKLQVKNAEGKWTELTGDERKKALDTLKSMKSISLDDHDAKTDMLISKYKSEKDRLAQEIVGVITGSAMPGGSANRIPNKAGSNPEGSIATRFIAETMYNELKTVDLTIQNAGGVRADILPGNVTFNDAYTFLPFGNTLYTYKMEGSLVKQVLEDAMQFALVDGSTGAFPYGAGIRYEANETPNAEGKRLVSVEVLNKQTQQWEPIDDNKRYLVGTNAYVAGGKDGYKTFGKLFNDPKYEGVDTYLPDAESFIKFMKKHPHFEAYTSSNVKFNASTDALPKK

SEQ ID NO: 4 - protein, 752 aa, Escherichia coli
MRVDAIAKVTGRARYTDDYVMAGMCYAKYVRSPIAHGYAVSINDEQARSLPGVLAIFTWEDVPDIPFATAGHAWTLDENKRDTADRALLTRHVRHHGDAVAIVVARDELTAEKAAQLVSIEWQELPVITTPEAALAEDAAPIHNGGNLLKQSTMSTGNVQQTIDAADYQVQGHYQTPVIQHCHMESVTSLAWMEDDSRITIVSSTQIPHIVRRVVGQALDIPWSCVRVIKPFVGGGFGNKQDVLEEPMAAFLTSKLGGIPVKVSLSREECFLATRTRHAFTIDGQMGVNRDGTLKGYSLDVLSNTGAYASHGHSIASAGGNKVAYLYPRCAYAYSSKTCYTNLPSAGAMRGYGAPQVVFAVESMLDDAATALGIDPVEIRLRNAAREGDANPLTGKRIYSAGLPECLEKGRKIFEWEKRRAECQNQQGNLRRGVGVACFSYTSNTWPVGVEIAGARLLMNQDGTINVQSGATEIGQGADTVFSQMVAETVGVPVSDVRVISTQDTDVTPFDPGAFASRQSYVAAPALRSAALLLKEKIIAHAAVMLHQSAMNLTLIKGHIVLVERPEEPLMSLKDLAMDAFYHPERGGQLSAESSIKTTTNPPAFGCTFVDLTVDIALCKVTINRILNVHDSGHILNPLLAEGQVHGGMGMGIGWALFEEMIIDAKSGVVRNPNLLDYKMPTMPDLPQLESAFVEINEPQSAYGHKSLGEPPIIPVAAAIRNAVKMATGVAINTLPLTPKRLYEEFHLAGLI

SEQ ID NO: 5 - protein, 292 aa, Escherichia coli
MFDFASYHRAATLADAINLLADNPQAKLLAGGTDVLIQLHHHNDRYRHIVDIHNLAELRGITLAEDGSLRIGSATTFTQLIEDPITQRHLPALCAAATSIAGPQIRNVATYGGNICNGATSADSATPTLIYDAKLEIHSPRGVRFVPINGFHTGPGKVSLEHDEILVAFHFPPQPKEHAGSAHFKYAMRDAMDISTIGCAAHCRLDNGNFSELRLAFGVAAPTPIRCQHAEQTAQNAPLNLQTLEAISESVLQDVAPRSSWRASKEFRLHLIQTMTKKVISEAVAAAGGKLQ

SEQ ID NO: 6 - protein, 159 aa, Escherichia coli
MNHSETITIECTINGMPFQLHAAPGTPLSELLREQGLLSVKQGCCVGECGACTVLVDGTAIDSCLYLAAWAEGKEIRTLEGEAKGGKLSHVQQAYAKSGAVQCGFCTPGLIMATTAMLAKPREKPLTITEIRRGLAGNLCRCTGYQMIVNTVLDCEKTK

SEQ ID NO: 7 - protein, 282 aa, Cellulomonas sp.
TTTTPPSTPPLDDPATDPFLVARAAADHIAQATGVEGHDMALVLGSGWGGAAELLGEVVAEVPTHEIPGFSAPAVAGHLSVTRSIRVERADGSVRHALVLGSRTHLYEGKGVRAVVHGVRTAAATGAETLILTNGCGGLNQEWGAGTPVLLSDHINLTARSPLEGPTFVDLTDVYSPRLRELAHRVDPTLPEGVYAQFPGPHYETPAEVRMAGILGADLVGMSTTLEAIAARHCGLEVLGVSLVTNLAAGISPTPLSHAEVIEAGQAAGPRISALLADIAKR

SEQ ID NO: 8 - protein, 490 aa, Leuconostoc mesenteroides
MEIQNKAMLITYADSLGKNLKDVHQVLKEDIGDAIGGVHLLPFFPSTGDRGFAPADYTRVDAAFGDWADVEALGEEYYLMFDFMINHISRESVMYQDFKKNHDDSKYKDFFIRWEKFWAKAGENRPTQADVDLIYKRKDKAPTQEITFDDGTTENLWNTFGEEQIDIDVNSAIAKEFIKTTLEDMVKHGANLIRLDAFAYAVKKVDTNDFFVEPEIWDTLNEVREILTPLKAEILPEIHEHYSIPKKINDHGYFTYDFALPMTTLYTLYSGKTNQLAKWLKMSPMKQFTTLDTHDGIGVVDARDILTDDEIDYASEQLYKVGANVKKTYSSASYNNLDIYQINSTYYSALGNDDAAYLLSRVFQVFAPGIPQIYYVGLLAGENDIALLESTKEGRNINRHYYTREEVKSEVKRPVVANLLKLLSWRNESPAFDLAGSITVDTPTDTTIVVTRQDENGQNKAVLTADAANKTFEIVENGQTVMSSDNLTQN

SEQ ID NO: 9 - protein, 178 aa, Escherichia coli
MKHTVEVMIPEAEIKARIAELGRQITERYKDSGSDMVLVGLLRGSFMFMADLCREVQVSHEVDFMTASSYGSGMSTTRDVKILKDLDEDIRGKDVLIVEDIIDSGNTLSKVREILSLREPKSLAICTLLDKPSRREVNVPVEFIGFSIPDEFVVGYGIDYAQRYRHLPYIGKVILLDE

SEQ ID NO: 10 - protein, 491 aa, Homo sapiens [sequence omitted]

SEQ ID NO: 11 - protein, 176 aa, Escherichia coli
MSLLNVPAGKDLPEDIYVVIEIPANADPIKYEIDKESGALFVDRFMSTAMFYPCNYGYINHTLSLDGDPVDVLVPTPYPLQPGSVIRCRPVGVLKMTDEAGEDAKLVAVPHSKLSKEYDHIKDVNDLPELLKAQIAHFFEHYKDLEKGKWVKVEGWENAEAAKAEIVASFERAKNK

SEQ ID NO: 12 - protein, 462 aa, Synechocystis sp. PCC 6803
MNTNLILDVDSYKVSHWLQYPPDTTAMYSYVESRGGRYPVTVFFGLQYILKRYLTQSIEPWMVEEANRLLTAHGLPFNYGGWRYIAEDLQGRLPVRIKAVPEGSVIPVHNVLMTVESTDPKVFWLVSWLETLLMRVWYPITVATQSWHLKQRIYQSLCRTADDPDGEINFKLHDFGARGVSSGESSGIGGLAHLVNFQGSDTVKALVYGQQYYNCPMAAYSIPAAEHSTITAWGREGEVLAYENMLTQFAKPGSVLAVVSDSYDLWNAIDHLWGDHLRAQVLDSGATVVIRPDSGDPVAIVAQTLERLEACFGSTLNSKGFRVLNAVRVIQGDGVDEESISAILEKTESLGFSTTNLAFGMGGALLQKVNRDTQKFAMKCSEVTVEDKAIPVYKDPVTDPGKTSKKGRLSLVKTDSGYGTVPTSSEDLLQVVYENGHLLQDQCLDAIRQRAWPLIRVNVPAS